(19) United States
(12) Patent Application Publication
(10) Pub. No.: US 2020/0253979 A1
(43) Pub. Date: Aug. 13, 2020

(54) THERAPEUTIC METHODS RELATING TO HSP90 INHIBITORS

(71) Applicant: AI Therapeutics, Inc., Guilford, CT (US)

(72) Inventors: Henri Lichenstein, Guilford, CT (US); Neil Beeharry, Guilford, CT (US); Sean Landrette, Meriden, CT (US); Sophia Gayle, East Haven, CT (US); Jeff Grotzke, Guilford, CT (US); Marylens Hernandez, Guilford, CT (US); Peter R. Young, Guilford, CT (US); Jonathan M. Rothberg, Guilford, CT (US)

(21) Appl. No.: 16/782,508

(22) Filed: Feb. 5, 2020

Related U.S. Application Data
(63) Continuation of application No. 16/144,198, filed on Sep. 27, 2018, now abandoned.
(60) Provisional application No. 62/563,991, filed on Sep. 27, 2017.
(60) Provisional application No. 62/587,886, filed on Nov. 17, 2017.
(60) Provisional application No. 62/688,079, filed on Jun. 21, 2018.

(51) Int. Cl. A61K 31/52; A61K 31/553; A61K 9/00; A61P 35/00; A61K 31/506; A61K 31/4725; A61K 31/337; A61K 31/5377; A61K 31/4709; A61K 31/095; A61K 31/357; A61K 31/4184; A61K 31/496; A61K 31/7068; A61K 31/435; A61K 31/704; A61K 45/06

(52) U.S. Cl. CPC A61K 31/52; A61K 45/06; A61K 9/0019; A61P 35/00; A61K 31/506; A61K 31/4725; A61K 31/337; A61K 31/5377; A61K 31/4709; A61K 31/095; A61K 31/357; A61K 31/4184; A61K 31/496; A61K 31/7068; A61K 31/435; A61K 31/704; A61K 31/553

(57) ABSTRACT

The disclosure provides methods for treating cancer, including but not limited to, hematopoietic and lung cancers, using the HSP90 inhibitor, MPC-0767, as monotherapy and in combination therapy with additional active agents, including but not limited to, inhibitors of Bcl-2, EZH2 inhibitors, Ras/Raf/MEK/ERK pathway inhibitors, checkpoint inhibitors, DNMT inhibitors, ATO and chemotherapeutic agents. The disclosure also provides related compositions and methods of use.

### FIELD OF THE INVENTION

The invention relates to the use of HSP90 inhibitors for the treatment of cancer.

### BACKGROUND OF THE INVENTION

Heat shock proteins (HSPs) are a class of chaperone proteins that are involved in diverse cellular processes such as elevation in temperature, external stresses, and nutrient deprivation. Their basic role as chaperone proteins is to stabilize proteins under such stresses but also to facilitate the correct folding of client proteins.

HSP90 is a highly conserved, ubiquitously expressed, molecular chaperone that plays an important role in regulating post-translational folding, stability, and function of cellular proteins (often referred to as “client proteins”), particularly in response to stress (Whitesell and Lindquist, Nature Rev. Cancer 2005 5:761). Folding of client proteins is dependent on the ATPase activity of HSP90, and inhibitors of HSP90 that bind to the ATP site can result in degradation of client proteins through the ubiquitin-proteasome pathway.

HSP90 is prominently involved in cancer due to its client proteins which include various oncogenes. (See e.g., Shrestha et al., 2016). Some client proteins are particularly responsive to HSP90 inhibitors and undergo rapid degradation. (Biamonte et al. J. Med. Chem 2010 53, 3-17). The most sensitive client proteins include HER2, wild-type EGFR and mutant EGFR, RAF-1, AKT, mutant BRAF, FLT3 and mutant FLT3.

Expression of HSP90 is often elevated in tumors (Valbuena et al., Mod. Pathology 2005 18: 1343; Guo et al., 2017), and has been associated with a poor prognosis (Pick et al., Cancer Res. 2007; Wang, J. et al., PLoS One 2013 8: e62876). Many tumor cells also express mutated or altered forms of proteins that are known to drive tumor growth, and these proteins are stabilized through association with HSP90 and dependent on this association for function. This association leads to the formation of a large protein complex within cells, which has enhanced affinity for HSP90 inhibitors (Goldstein et al., J. Clin. Invest. 2015 125(12): 4559-71; Rodina et al., Nature 2016 538: 397). Consequently, tumor cells retain higher levels of HSP90 inhibitor and administration of HSP90 inhibitors results in potent client protein degradation and decreased proliferation and survival with more limited activity on normal cells (Barrott and Haystead, FEBS J. 2013 280:1381).

HSP90 inhibitors have been tested in pre-clinical and early clinical studies relating to various cancers including breast, colorectal, gastro-intestinal, leukemia, lymphomas, melanoma, multiple myeloma, ovarian, pancreatic, prostate and renal. At least 18 HSP90 inhibitors have been investigated in clinical trials, including BIIB021, IPI-493, MPC-3100, Debio0932, DS-2248, HSP990, XL888, SNX5422, TAS-116, BIIB028, IPI-504, KW-2478, alvespimycin, tanespimycin, AT13387, AUY922, PU-H71 and ganetespib. See reviews by Bhat et al., J. Med. Chem 2014 57:8718-8728; Neckers and Workman Clin. Cancer Res. 2012, 18, 64. To date, none of these compounds have been approved for use in humans, and no HSP90 inhibitor has been tested in a genetically defined population.

Emerging evidence suggests that HSP90 may also affect tumor immunity. Some non-clinical studies have suggested that high HSP90 inhibitor doses may inhibit various components of the immune system that may be important for tumor clearance (Bae et al., J. Immunol. 2007 178: 7730; Bae et al., J. Immunol. 2013 190:1360; Tukaj et al., J Inflammation 2014 11:10). In addition, many tumor cells express the checkpoint inhibitor protein death ligand 1 (PD-L1) in their surface, which can suppress local cytotoxic T cell activity. For example, PD-L1 expression is found on patient AML cells, increases with disease progression and during relapse (Salih et al., Exp. Hematol. 2006 34:888; Chen et al., Cancer Biol. Ther. 2008 7:622; Berthon et al., Cancer Immunol. Immunother 2010 59:1839) and is associated with poorer overall survival (Brodska et al., Blood 2016 128:5229). PD-L1 cell surface expression on AML tumor cells may be induced by IFN-γ which is known to be expressed in the immunologically active tumor microenvironment (Berthon et al, Cancer Immunol. Immunother. 2010 59:1839; Kronig et al., Eur. J. Hematol. 2013 92:195).

There is a continuing need for improved treatments and drug combinations for treating cancer, particularly in the treatment of cancers that are refractory to current therapies, or those that have relapsed after treatment, such as those based on protein tyrosine kinase inhibitors. The present invention addresses this need with the use of HSP90 inhibitors.

### SUMMARY OF THE INVENTION

The disclosure provides compositions and methods related to the use of an HSP90 inhibitor for treating cancer in a subject, preferably a human subject, in need of such treatment. The methods relate generally to the use of MPC-0767 in the treatment of cancer, and more particularly in treating a cancer whose cell growth and/or survival is characterized as driven by or dependent upon activated protein kinase signaling pathways, and/or a cancer which is refractory to, or which has relapsed after, treatment with a therapeutic agent. As described in more detail infra, MPC-0767 demonstrates potent anti-cancer activity against certain cancers when used alone, and also demonstrates surprising efficacy in combination with other therapeutic agents.

The disclosure provides methods for treating cancer in a subject in need thereof, comprising administering to the subject a pharmaceutical composition comprising a therapeutically effective amount of MPC-0767, or a pharmaceutically acceptable salt thereof, and optionally a pharmaceutically acceptable carrier or excipient. In embodiments, the pharmaceutical composition comprises a mesylate salt of MPC-0767. In embodiments, the pharmaceutical composition comprises a salt of MPC-0767 selected from a hydrochloride, hydrobromide, sulfate, phosphate, fumarate, succinate, or maleate salt. In embodiments, the subject in need of treatment is one whose cancer is refractory to treatment with, or has relapsed after treatment with, at least one therapeutic agent. In embodiments, the cancer is refractory to, or has relapsed after, treatment with at least one therapeutic agent. In embodiments, the therapeutic agent is a protein kinase inhibitor. In embodiments, the therapeutic agent is a Bcl-2 inhibitor or a Bcl-2 pathway inhibitor. In embodiments, the therapeutic agent is selected from erlotinib, afatinib, lapatinib, dacomitinib, gefitinib, AP32788, poziotinib, osimertinib and EGF816. In other embodiments, the therapeutic agent is selected from gilteritinib, tandutinib, crenolanib, sorafenib, midostaurin, and quizartinib. In embodiments, the therapeutic agent is gilteritinib. In embodiments, the therapeutic agent is midostaurin. In embodiments, the therapeutic agent is sorafenib. In embodiments, the therapeutic agent is tandutinib.

In embodiments, the cancer is characterized as having one or more activating mutations in at least one protein kinase selected from epidermal growth factor receptor (EGFR), human epidermal growth factor receptor 2 (HER2), and fms-like tyrosine kinase 3 (FLT3). In embodiments, the one or more activating mutations is an EGFR or HER2 exon 20 insertion mutation (ins20). In embodiments, the one or more activating mutations is an FLT3 internal tandem duplication (ITD).

In embodiments, the cancer is a hematologic malignancy or a solid tumor.

In embodiments, the cancer is selected from gastric cancer, colon cancer, prostate cancer, small-cell lung cancer, non-small cell lung cancer (NSCLC), ovarian cancer, lymphoma, acute myeloid leukemia (AML), chronic lymphocytic leukemia (CLL), multiple myeloma, renal cell carcinoma, gastrointestinal stromal tumor, chronic myeloid leukemia, glioblastoma multiforme, astrocytomas, medulloblastomas, melanoma, breast cancer, and pancreatic cancer. In embodiments, the cancer is NSCLC. In embodiments, the cancer is AML. In embodiments, the cancer is CLL. In embodiments, the cancer is characterized as having one or more activating mutations in at least one protein kinase selected from EGFR and HER and the cancer is NSCLC. In embodiments, the cancer is characterized as having one or more activating mutations in FLT3 and the cancer is AML.

In accordance with any of the preceding embodiments, the subject is human.

In accordance with any of the preceding embodiments, the pharmaceutical composition is adapted for oral, buccal, or parenteral administration.

In accordance with any of the preceding embodiments, the method further comprises administering to the subject one or more additional active pharmaceutical ingredients (APIs).

In embodiments, the one or more additional APIs is a protein kinase inhibitor (PKI), an FLT3 inhibitor, a PD-1/PD-L1 inhibitor, a CTLA-4 inhibitor, a Ras/Raf/MEK/ERK pathway inhibitor, a Bcl-2 pathway inhibitor, or an EZH2 inhibitor.

In embodiments, the one or more additional APIs is a PKI. In embodiments, the PKI is an EGFR or HER2 targeted PKI. In embodiments, the PKI is selected from erlotinib, afatinib, lapatinib, dacomitinib, gefitinib, AP32788, poziotinib, osimertinib and EGF816. In accordance with any of the embodiments where the API is a PKI, in another embodiment the cancer is NSCLC.

In embodiments, the one or more additional APIs is an FLT3 inhibitor. In embodiments, the FLT3 inhibitor is selected from tandutinib, crenolanib, gilteritinib, midostaurin, quizartinib, and sorafenib. In accordance with any of the embodiments where the API is an FLT3 inhibitor, in another embodiment the cancer is AML.

In embodiments, the one or more additional APIs is a PD-1/PD-L1 inhibitor. In embodiments, the PD-1/PD-L1 inhibitor is selected from the group consisting of AMP-224, AMP-514/MEDI-0680, atezolizumab, avelumab, BGB-A317, BMS936559, durvalumab, JTX-4014, nivolumab, pembrolizumab, and SHR-1210. In accordance with any of the embodiments where the API is a PD-1/PD-L1 inhibitor, in another embodiment the cancer is AML.

In embodiments, the Ras/Raf/MEK/ERK pathway inhibitor is trametinib

In embodiments, the one or more additional APIs is a Bcl-2 pathway inhibitor. In embodiments, the Bcl-2 pathway inhibitor is selected from the group consisting of ABT-737, AT-101 (Gossypol), APG-1252, A1155463, A1210477, navitoclax, obatoclax, sabutoclax, venetoclax, S 55746, WEHI-539, AMG-176, MIK665 and S641315. In embodiments, the Bcl-2 pathway inhibitor is an inhibitor of BCL2, BCLXL, or MCL1. In embodiments, the Bcl-2 pathway inhibitor is selected from ABT-737, navitoclax, and venetoclax, preferably venetoclax. In accordance with any of the embodiments where the API is a Bcl-2 pathway inhibitor, in another embodiment the cancer is AML or CLL.

In embodiments, the one or more additional APIs is an EZH2 inhibitor. In embodiments, the EZH2 inhibitor is selected from EPZ6438, CPI-1205, GSK343, GSK2816126, MAK-683 and PF-06821497.

In embodiments, the one or more additional APIs is a chemotherapeutic agent. In embodiments, the chemotherapeutic agent is selected from arsenic trioxide or azacytidine.

In embodiments, the chemotherapeutic agent is selected from docetaxel, carboplatin, cisplatin, and pemetrexed. In an embodiment where the API is a chemotherapeutic agent, the cancer is NSCLC.

In embodiments, the one or more additional APIs is selected from daunorubicin, doxorubicin, epirubicin, mitoxantrone, idarubicin, and cytarabine. In embodiments where the one or more additional APIs is selected from daunorubicin, doxorubicin, epirubicin, mitoxantrone, idarubicin, and cytarabine, the cancer is AML.

In embodiments, the one or more additional APIs is selected from crenolanib, cytarabine, daunorubicin, gilteritinib, sorafenib, and venetoclax. In embodiments where the one or more additional APIs is selected from crenolanib, cytarabine, daunorubicin, gilteritinib, sorafenib, and venetoclax, the cancer is AML.

The disclosure also provides methods for treating acute myelogenous leukemia (AML) in a subject in need thereof, the method comprising administering to the subject a pharmaceutical composition comprising a therapeutically effective amount of MPC-0767, or a pharmaceutically acceptable salt thereof, and optionally a pharmaceutically acceptable carrier or excipient. In embodiments, the pharmaceutical composition comprises a mesylate salt of MPC-0767. In embodiments, the pharmaceutical composition comprises a salt of MPC-0767 selected from a hydrochloride, hydrobromide, sulfate, phosphate, fumarate, succinate, or maleate salt. In embodiments, the AML is refractory to, or has relapsed after, treatment with at least one protein kinase inhibitor (PKI). In embodiments, the AML is refractory to, or has relapsed after, treatment with one or more of midostaurin, quizartinib and sorafenib. In embodiments, the AML is refractory to, or has relapsed after, treatment with one or more of gilteritinib, crenolanib, sorafenib, midostaurin, daunorubicin, doxorubicin, epirubicin, mitoxantrone, idarubicin, and cytarabine. In embodiments, the AML is characterized as having one or more activating mutations in FLT3. In embodiments, the one or more activating mutations in FLT3 is selected from the FLT3 ITD mutation, a point mutation at FLT3 D835, a point mutation at FLT3 I836, the point mutation FLT3 N676K, and the point mutation F691L. In embodiments, the one or more activating mutations in FLT3 is the FLT3 ITD mutation.

In an embodiment, the AML is characterized as wild-type for FLT3 and without an activating Ras mutation.

In embodiments, the methods for treating AML further comprise a step of administering one or more additional active pharmaceutical agents (APIs) to the subject. In embodiments, the one or more additional APIs is a protein kinase inhibitor (PKI), a chemotherapeutic agent, an FLT3 inhibitor, a PD-1/PD-L1 inhibitor, a Bcl-2 pathway inhibitor, or an EZH2 inhibitor. In embodiments, the FLT3 inhibitor is selected from tandutinib, crenolanib, gilteritinib, midostaurin, quizartinib, and sorafenib. In embodiments, the PD-1/PD-L1 inhibitor is selected from AMP-224, AMP-514/MEDI-0680, atezolizumab, avelumab, BGB-A317, BMS936559, durvalumab, JTX-4014, nivolumab, pembrolizumab, and SHR-1210. In embodiments, the Bcl-2 pathway inhibitor is selected from ABT-737, AT-101 (Gossypol), APG-1252, A1155463, A1210477, navitoclax, obatoclax, sabutoclax, venetoclax, S 55746, WEHI-539, AMG-176, MIK665 and S641315. In embodiments, the Bcl-2 pathway inhibitor is an inhibitor of BCL2, BCLXL, or MCL1. In embodiments, the Bcl-2 pathway inhibitor is selected from ABT-737, navitoclax, and venetoclax. In embodiments, the EZH2 inhibitor is selected from EPZ6438, CPI-1205, GSK343, GSK2816126, MAK-683 or PF-06821497.

In embodiments, the one or more additional APIs is selected from daunorubicin, doxorubicin, epirubicin, mitoxantrone, idarubicin, and cytarabine.

In embodiments, the one or more additional APIs is selected from crenolanib, cytarabine, daunorubicin, gilteritinib, sorafenib, and venetoclax.

In embodiments, the one or more additional APIs is venetoclax.

In embodiments, the one or more additional APIs is a Raf/Ras/MAPK pathway inhibitor.

In embodiments, the one or more additional APIs is a chemotherapeutic agent selected from arsenic trioxide (ATO), azacytidine, and decitabine.

The disclosure also provides a pharmaceutical composition comprising MPC-0767, or a pharmaceutically acceptable salt thereof, and optionally a pharmaceutically acceptable carrier or excipient.

The disclosure also provides a pharmaceutical composition comprising MPC-0767, or a pharmaceutically acceptable salt thereof, and optionally a pharmaceutically acceptable carrier or excipient for use in treating AML according to the methods described herein.

The disclosure also provides a pharmaceutical composition comprising MPC-0767 and one or more additional APIs. In embodiments, the one or more additional APIs is selected from crenolanib, cytarabine, daunorubicin, gilteritinib, sorafenib, and venetoclax. In embodiments, the one or more additional APIs is selected from ABT-737, navitoclax, and venetoclax. In embodiments, the one or more additional APIs is venetoclax.

In embodiments, the disclosure provides methods for treating acute myelogenous leukemia (AML) in a subject in need thereof, comprising administering to the subject a pharmaceutical composition comprising a therapeutically effective amount of MPC-0767, or a pharmaceutically acceptable salt thereof, preferably a mesylate salt, and optionally a pharmaceutically acceptable carrier or excipient, wherein the AML is refractory to, or has relapsed after, treatment with a Bcl-2 pathway inhibitor. In embodiments, the AML has relapsed after treatment with venetoclax. In embodiments, the method further comprises administering one or more additional active pharmaceutical agents (APIs) to the subject. In embodiments, the one or more additional APIs is selected from a protein kinase inhibitor (PKI), a chemotherapeutic agent, an FLT3 inhibitor, a PD-1/PD-L1 inhibitor, and a Bcl-2 pathway inhibitor. In embodiments, the FLT3 inhibitor is selected from crenolanib, gilteritinib, midostaurin, quizartinib, and sorafenib. In embodiments, the PD-1/PD-L1 inhibitor is selected from the group consisting of AMP-224, AMP-514/MEDI-0680, atezolizumab, avelumab, BGB-A317, BMS936559, durvalumab, JTX-4014, nivolumab, pembrolizumab, and SHR-1210. In embodiments, the Bcl-2 pathway inhibitor is selected from the group consisting of ABT-737, AT-101 (Gossypol), APG-1252, A1155463, A1210477, navitoclax, obatoclax, sabutoclax, venetoclax, S 55746, WEHI-539, AMG-176, MIK665 and 5641315. In embodiments, the Bcl-2 pathway inhibitor is an inhibitor of BCL2, BCLXL, or MCL1. In embodiments, the Bcl-2 pathway inhibitor is selected from ABT-737, navitoclax, and venetoclax. In embodiments, the one or more additional APIs is selected from the group consisting of daunorubicin, doxorubicin, epirubicin, mitoxantrone, idarubicin, and cytarabine. In embodiments, the one or more additional APIs is selected from crenolanib, cytarabine, daunorubicin, gilteritinib, sorafenib, and venetoclax. In embodiments, the one or more additional APIs is venetoclax.

In embodiments, the disclosure provides methods for treating acute myelogenous leukemia (AML) in a subject in need thereof, the methods comprising administering to the subject a pharmaceutical composition comprising a therapeutically effective amount of MPC-0767, or a pharmaceutically acceptable salt thereof, preferably a mesylate salt, and optionally a pharmaceutically acceptable carrier or excipient, in a combination therapy regimen further comprising administering a Ras/Raf/MEK/ERK pathway inhibitor. In embodiments, the Ras pathway inhibitor is selected from a Raf inhibitor such as vemurafenib, sorafenib, or dabrafenib, a MEK inhibitor such as AZD6244 (Selumetinib), PD0325901, GSK1120212 (Trametinib), U0126-EtOH, PD184352, RDEA119 (Rafametinib), PD98059, BIX 02189, MEK162 (Binimetinib), AS-703026 (Pimasertib), SL-327, BIX02188, AZD8330, TAK-733, cobimetinib or PD318088, and an ERK inhibitor such as LY3214996, BVD-523 or GDC-0994.

In embodiments, the disclosure provides methods for treating acute myelogenous leukemia (AML) in a subject in need thereof, the methods comprising administering to the subject a pharmaceutical composition comprising a therapeutically effective amount of MPC-0767, or a pharmaceutically acceptable salt thereof, preferably a mesylate salt, and optionally a pharmaceutically acceptable carrier or excipient, in a combination therapy regimen further comprising administering an EZH2 inhibitor such as EPZ6438, CPI-1205, GSK343, GSK2816126, MAK-683 or PF-06821497.

In embodiments, the disclosure provides methods for treating acute myelogenous leukemia (AML) in a subject in need thereof, the methods comprising administering to the subject a pharmaceutical composition comprising a therapeutically effective amount of MPC-0767, or a pharmaceutically acceptable salt thereof, preferably a mesylate salt, and optionally a pharmaceutically acceptable carrier or excipient, in a combination therapy regimen further comprising administering a chemotherapeutic agent selected from arsenic trioxide (ATO), azacytidine, and decitabine.

In embodiments, the disclosure provides a pharmaceutical composition comprising MPC-0767, or a pharmaceutically acceptable salt thereof, preferably a mesylate salt, and optionally a pharmaceutically acceptable carrier or excipient, for use in treating AML according to any of the methods of MPC-0767 monotherapy or combination therapy described herein.

The disclosure also provides methods for predicting therapeutic response to MPC-0767 in a subject in need of treatment for AML, the method comprising determining the FLT3 and RAS status in a sample of AML cancer cells obtained from the subject, wherein a status of FLT3 normal/non-FLT3-ITD and RAS mutant indicates that the cancer cells are predicted to be resistant to MPC-0767 monotherapy and responsive to a combination therapy with MPC-0767 and a Ras/Raf/MEK/ERK pathway inhibitor; and a status of FLT3-ITD indicates that the cancer cells are predicted to be responsive to MPC-0767 monotherapy.

The disclosure also provides methods for treating AML in a subject in need of such treatment, the method comprising determining the FLT3 and RAS mutant status in a sample of AML cancer cells from the subject and treating the subject with a combination therapy comprising MPC-0767 and a Ras/Raf/MEK/ERK pathway inhibitor where the status is FLT3 normal or non-FLT3-ITD and RAS mutant.

In accordance with the foregoing methods, a status of Ras mutant may be defined by the presence of one or more activating mutations in NRAS or KRAS. In embodiments, the one or more activating mutations in NRAS or KRAS is a mutation in the polynucleotide sequence encoding the RAS protein that results in an amino acid change selected from the group consisting of A146T and G13D of KRAS; or selected from Q61L, Q61H, and G12D of NRAS.

The disclosure also provides methods for predicting therapeutic response to MPC-0767 in a subject in need of treatment for AML, the method comprising determining or receiving the EZH2 status in a sample of AML cancer cells from the subject, wherein an EZH2 loss of function mutation indicates that the cancer cells are predicted to be responsive to MPC-0767 therapy while an EZH2 gain of function mutation indicates that the cancer cells are predicted to be resistant to MPC-0767 therapy. In embodiments, the MPC-0767 therapy is monotherapy or combination therapy.

The disclosure also provides methods for treating AML in a subject in need of such treatment, the method comprising determining or receiving the EZH2 status of the AML in a biological sample of the AML from the subject and treating the subject with MPC-0767 therapy where the status is an EZH2 loss of function mutation, or treating the subject with a combination therapy comprising MPC-0767 and an EZH2 inhibitor where the EZH2 status is normal or a gain of function EZH2 mutation. In embodiments, the MPC-0767 therapy is monotherapy or combination therapy.

The disclosure also provides methods for predicting therapeutic response to MPC-0767 in a subject in need of treatment for AML, the method comprising determining or receiving the KDM6A status in a sample of AML cancer cells obtained from the subject, wherein a KDM6A loss of function mutation indicates that the cancer cells are predicted to be resistant to MPC-0767 therapy. In embodiments, the MPC-0767 therapy is monotherapy or combination therapy.

### BRIEF DESCRIPTION OF THE FIGURES

FIG. 1A-D: MPC-0767 inhibits viability of non-small cell lung cancer cell lines having mutations in EGFR or HER2. FIG. 1A; HCC-827; FIG. 1B: H1975; FIG. 1C: PC-9; FIG. 1D: H1781.

FIG. 2: MPC-0767 induces cell death in H1975 cells.

FIG. 3A-B: MPC-0767 reduces cell surface EGFR expression in H1975 cells (A) and PC-9 cells (B). Cells were treated with MPC-0767 (1 μM) for 24 hours before being harvested and cell expression of EGFR determined by flow cytometry.

FIG. 4A-B: MPC-0767 dose-dependently reduces (A) cell surface expression of EGFR WT and EGFR Exon20 mutant (V769_D770insASV) in BaF3 cells after 24 hours treatment and (B) cell viability of parental BaF3 or BaF3 expressing EGFR Exon20 mutant (V769_D770insASV) after 72 hours treatment.

FIG. 5A-C: MPC-0767 has cytotoxic activity in AML cells harboring FLT3-ITD. Cell viability of (A) FLT3 wild-type cells, ME-1, and (B) MV-4-11 cells harboring FLT3-ITD, (C) summary data showing EC₅₀ values from AML cell lines and primary AML cells treated for 72 hours.

FIG. 6: MPC-0767 induces dose-dependent cell death in primary AML cells harboring FLT3-ITD after 72 hours treatment. Sample Y1265 was obtained from a patient whose AML had relapsed after treatment with gilteritinib.

FIG. 7A-B: MPC-0767 demonstrates antitumor activity in a mouse xenograft model of AML FLT3-ITD (MV-4-11 cells). Seven days post-tumor inoculation, mice (n=10 per group) were orally administered with vehicle alone, or MPC-0767 200 mg/kg QD×2 days then reduced to 150 mg/kg QD×15 days. Tumor size (mm³) (A) and body weight change (B) are shown. Five tumor regressions were found in the MPC-0767 treatment group and significance was found with the treatment, P<0.0001 (Student t-test).

FIG. 8A-C: AML FLT3-ITD cells generated to be resistant to midostaurin cytotoxicity (MOLM-13-R-PKC412, black line in each graph) are resistant to midostaurin, 2-100 nM (A) and crenolanib, 0.2-100 nM (B), but not to MPC-0767, 20-10000 nM (C). Grey line in each graph is MOLM-13-LUC. Cells were treated for 72 hours before viability was assessed.

FIG. 9A-C: MPC-0767 retains cytotoxic activity under stromal conditions which confer resistance to FLT3 inhibitors. MOLM-14 cells were treated with gilteritinib (A), crenolanib (B), or MPC-0767 (C) in either non-stromal media (black lines in each graph) or stromal media (grey lines in each graph). Cells were treated for 72 hours before viability was assessed.

FIG. 10A-D: MPC-0767 reduces cell surface expression of FLT3 (A, B) and subsequently reduces phosphorylation of the downstream target S6 (10C, 10D). MV-4-11 cells (A, C) or MOLM-13 cells (B, D) are treated with vehicle or MPC-0767 for 24 hours.

FIG. 11A-C: MPC-0767 ablates cell surface expression of transfected wild type and mutant FLT3 in BaF3 cells (A). In a cytotoxicity assay, an engineered BaF3 cell line expressing FLT3-ITD (grey line in each graph) and with a F691L mutation (black line in each graph) is resistant to crenolanib (B) but remains sensitive to MPC-0767 (C).

FIG. 12: MPC-0767 reduces interferon-gamma-induced PD-L1 cell surface expression in six primary AML patient samples. Cells were treated with human IFN-γ (50 ng/ml) and/or MPC-0767 (1 μM) for 24 hours.

FIG. 13A-E: MPC-0767 shows synergistic cytotoxic activity in combination with daunorubicin (A), cytarabine (B), crenolanib (C), sorafenib (D), and venetoclax (E) in MV-4-11 cells.

FIG. 14: MPC-0767 shows potent anti-tumor activity in combination with venetoclax. A systemic survival xenograft study was performed using the MOLM-13 FLT3-ITD harboring AML cell line. Shown are survival curves for mice treated with vehicle (grey line), MPC-0767 (dashed line) 100-60 mg/kg QD, venetoclax (dotted line) 45-33.84 mg/kg QD, or the combination of MPC-0767 and venetoclax (solid line). Combination vs MPC-0767 alone, venetoclax alone, or vs vehicle alone P<0.001, Log Rank (Mantel Cox) test.

FIG. 15: EC₅₀ values of MPC-0767 (left four bars) or venetoclax (right four bars) in parental and venetoclax-resistant (Ven-R) MOLM-13 and MV-4-11 cells. Cells were treated with MPC-0767 or venetoclax for 72 h and cell viability was determined using a CTG assay. Experiments were performed a minimum of 2 independent times, in duplicate, and averaged data ±SD are shown.

FIG. 16A-B: (A) Western blot analysis of MV-4-11 venetoclax-resistant cells treated with MPC-0767 (580 nM), venetoclax (2500 nM) or the combination for 24 hours. Lysates were probed with antibodies to PARP and vinculin was used as a loading control. Upper and lower arrows denote full length PARP and cleaved PARP, respectively. Representative data shown from 2 independent experiments. (B) Normalized isobologram at the ED75 of two venetoclax-resistant cell lines treated with the combination of MPC-0767 and venetoclax for 72 hours before viability assayed using CellTiter-Glo®. Each data point is the average of 2 independent experiments, performed in duplicate, for each cell line.

FIG. 17A-B: (A) Western blot analysis of MOLM-14 cells treated with MPC-0767 (1 μM), venetoclax (20 nM) or the combination for 24 hours. Lysates were probed with the indicated antibodies. Vinculin was used as a loading control. Representative blot shown from 2 independent experiments. (B) Western blot analysis of MV-4-11 venetoclax-resistant cells treated with MPC-0767 (580 nM), venetoclax (2500 nM) or the combination for 24 hours. Lysates were probed with antibodies to AKT and MCL-1. Vinculin was used as a loading control. Representative data shown from 2 independent experiments.

FIG. 18: MPC-0767 sensitivity of AML cells harboring wild-type FLT3. Dot-plot of EC₅₀ values from AML cell lines and primary AML samples treated with MPC-0767 for 72 h followed by viability determination using CellTiter-Glo®. Experiments using cell lines were performed 2 independent times, each in duplicate, while primary AML blasts were assayed once, in duplicate. Geometric mean is shown by horizontal line.

FIG. 19A-B: CRISPR identifies epigenetic regulation as a key determinant of MPC-0767 sensitivity. (A) Gene ontology analysis of top 20 sgRNAs. (B) Scatter-plot showing enrichment of normalized sgRNA read count of KDM6A in vehicle and MPC-0767-treated CRISPR pools from the combined A and B GeCKO sublibaries. 6 individual sgRNAs used for targeting KDM6A are shown in black circles.

FIG. 20A-B: CRISPR-mediated targeting of KDM6A with three independent sgRNAs in the MOLM-14 and MV-4-11 cell lines confers resistance to MPC-0767. Viability of MOLM-14 (A) or MV-4-11 (B) cells with the indicated non-targeting sgRNA or KDM6A sgRNA treated with MPC-0767 (1 μM). After 72 hours treatment, cell viability was assessed using CTG. Data presented is the average of individual sgRNAs for each cell line±SD, performed twice, in duplicate.

FIG. 21: Normalized isobologram at the EC₇₅ of a FLT3-ITD harboring cell line (MV-4-11) treated with the EZH2 inhibitors EPZ6438 or CPI-1205 for 4 days followed by the combination of EZH2 inhibitors and MPC-0767 for an additional 72 hours before viability was assayed using CellTiter-Glo®. Each data point is the average of 3 independent experiments, each performed in duplicate, for each cell line.

FIG. 22: Bar graph showing the viability of MOLM-14 cells treated with MPC-0767 (527 nM), arsenic trioxide (ATO) (1250 nM) or the combination for 72 hours. Cl value determination confirmed the combination was synergistic (i.e., <1).

FIG. 23: Quantification of FLT3, pERK, pS6 levels in MOLM-13 cells treated with MPC-0767 (800 nM), ATO (625 nM) or the combination for 24 hours.

FIG. 24: EC₅₀ values of BaF3 cells expressing FLT3-ITD further supplemented with or without IL-3 and treated with the FLT3 inhibitors crenolanib and gilteritinib or with MPC-0767 for 72 hours. After this time cell viability was determined using CTG and EC₅₀ values determined. Graph is the average±SD of 2 independent studies, each performed in duplicate.

FIG. 25: MPC-0767 exhibits enhanced anti-tumor activity in combination with 5′azacitidine. A systemic survival xenograft study was performed using the MOLM-13 FLT3-ITD harboring AML cell line. Shown are survival curves for mice treated with vehicle (grey line), MPC-0767 (dashed line) 75 mg/kg (QDx5; 1 day off; QDx26), 5′azacitidine (dotted line) 2 mg/kg (QDx4), or the combination of MPC-0767 and 5′ azacitidine (solid line). Combination vs MPC-0767 alone, 5′ azacitidine alone, or vs vehicle alone P<0.001, Log Rank (Mantel Cox) test.

FIG. 26: OCI-AML2 cells pre-treated with MPC-0767 are more sensitive to T cell-mediated killing. DMSO was used as a vehicle control. Bars represent the mean +/−SD of 2 independent experiments.

FIG. 27A-D: MPC-0767 demonstrates antitumor activity in a mouse syngeneic model (MC38 cells). Eleven days post tumor inoculation, mice (n=6 per group) were orally administered with vehicle alone, or 150 mg/kg MPC-0767 QD x 17. Tumor size (mm³), P=0.01 (Student t-test) (A) and percent body weight change (B) are shown. (C) PD-L1 levels measured in MC38 tumor infiltrating leukocytes (CD45⁺, CD3⁻) after 7 days of 150 mg/kg MPC-0767 dosing, P<0.05 (Student t-test). (D) Ratio of CD4:TREG (Left) and CD8:TREG (Right) in MC38 tumors * P<0.05, ** P<0.01 (Student t-test). CD4 T-cells defined as CD45⁺, CD3⁺, CD4⁺; CD8 T-cells defined as CD45⁺, CD3⁺, CD4⁻, and TREGs defined as CD45⁺, CD3⁺, CD4⁺, FOXP3⁺.

FIG. 28: Bar graph showing the viability of MOLM-13 cells treated with MPC-0767 (351 nM), trametinib (25 nM) or the combination for 72 hours. CI value determination confirmed the combination was synergistic (i.e., <1).

FIG. 29A-D: MPC-0767 repression of PD-L1 expression increases T cell activation. Bar graphs show activation of Jurkat reporter cells with anti-CD3 (A) and PD-L1-dependent inhibition of T cell activation after IFNγ treatment (B). Bars in A&B represent the mean +/− SD of triplicate wells and are representative of three independent experiments. Bar graphs in C&D demonstrate MPC-0767 reduces cell surface expression of PD-L1 (C, p=0.0113 at 1 μM and <0.0001 at 2 μM compared to IFNγ alone) and also reduces inhibition of T cell activation (D, p=0.0198 at 1 μM and 0.0323 at 2 μM compared to IFNγ alone). Bars in C&D represent the mean +/− SD of three independent experiments.

FIG. 30: MPC-0767 demonstrates anti-tumor activity in a systemic in vivo AML model. Kaplan-Meier survival analysis of a MOLM-13 systemic model where mice were dosed orally with vehicle or with MPC-0767 (75 or 150 mg/kg daily). Statistical significance was calculated using Log Rank (Mantel-Cox) test. P<0.01 for MPC-0767 75 mg/kg and 150 mg/kg vs vehicle.

### DETAILED DESCRIPTION

The disclosure provides compositions and methods related to the use of MPC-0767, or a pharmaceutically acceptable salt thereof, for treating cancer in a subject, preferably a human subject, in need of such treatment.

WO 2011/060253 describes the parent compound of MPC-0767, MPC-3100, including its oral bioavailability in humans. MPC-3100 can be identified as (2S)-1-[4-(2-{6-Amino-8-[(6-bromo-1,3-benzodioxol-5-yl)sulfanyl]-9H-purin-9-yl}ethyl)piperidin-1-yl]-2-hydroxypropan-1-one and is described in Kim et al., J. Med. Chem. 2012 55, 7480-7501. As noted in a 2014 review, MPC-3100 is no longer in active development (Bhat et al. J. Med. Chem 2014 57:8718-8724). Although MPC-3100 successfully completed a phase I clinical study, its further clinical development was hindered by poor solubility (Kim et al. Bioorg. Med. Chem. Lett. 25:5254-5257) (2015). MPC-0767 is a pro-drug of MPC-3100 which was developed to address this problem with the parent compound. MPC-0767 showed improved aqueous solubility, adequate chemical stability, and rapid bioconversion. Id. MPC-0767 and related compounds are disclosed in WO 2012/148550, which is incorporated herein by reference in its entirety. MPC-0767 is converted into its parent compound primarily by an enzyme-mediated cleavage process. Its oral bioavailability when formulated in 2% carboxymethylcellulose was similar to that of the parent compound (40% Captisol™). MPC-0767 also showed similar efficacy as the parent compound in an N-87 xenograft tumor model. N-87 cells are human HER2 positive gastric cancer cells. The structure of MPC-0767 is shown below.

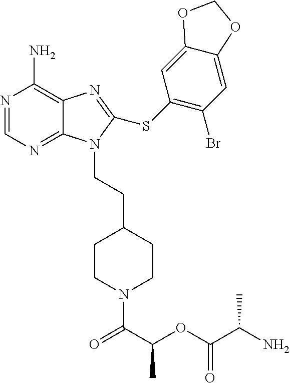 [SMILES: C[C@H](N)C(=O)O[C@@H](C)C(=O)N1CCC(CCn2c(Sc3cc4c(cc3Br)OCO4)nc3c(N)ncnc32)CC1]

In embodiments of the compositions and methods described here, the pharmaceutically acceptable salt of MPC-0767 is a mesylate salt. Accordingly, in embodiments, the disclosure provides methods of treating cancer in a subject, preferably a human subject, in need of such treatment, the methods comprising administering to the subject an effective amount of a mesylate salt of MPC-0767. In embodiments, the mesylate salt of MPC-0767 is in the form of a pharmaceutical composition. In embodiments, the pharmaceutical composition does not comprise a cyclodextrin. Pharmaceutical compositions and formulations comprising MPC-0767, and salts thereof, are described in more detail infra.

Both monotherapy and combination therapy methods of treating cancer with MPC-0767 are contemplated by the present disclosure. Combination therapies are discussed infra. In the context of MPC-0767 monotherapy, in some, but not all, embodiments the subject in need of treatment is one having a cancer that is non-responsive or refractory to, or has relapsed after, treatment with a ‘standard-of-care’ or first-line therapeutic agent. In this context, the terms “non-responsive” and “refractory” are used interchangeably herein and refer to the subject's response to therapy as not clinically adequate, for example to stabilize or reduce the size of one or more solid tumors, to slow tumor progression, to prevent, reduce or decrease the incidence of new tumor metastases, or to relieve one or more symptoms associated with the cancer. A cancer that is refractory to a particular drug therapy may also be described as a drug-resistant cancer. In a standard therapy for the cancer, refractory cancer includes disease that in progressing despite active treatment while “relapsed” cancer includes cancer that progresses in the absence of any current therapy, but following successful initial therapy.

Accordingly, in embodiments, the subject is one who has undergone one or more previous regimens of therapy with one or more ‘standard-of-care’ therapeutic agents. In such cases, the subject's cancer may be considered refractory or relapsed. In embodiments, the cancer is refractory to, or has relapsed after, treatment with a protein kinase inhibitor (PKI). In embodiments, the cancer is refractory to, or has relapsed after, treatment with a PKI targeted against one or more of the following kinases: breakpoint cluster region-Abelson (BCR-ABL), B-rapidly accelerated fibrosarcoma (B-RAF), epidermal growth factor receptor (EGFR), human epidermal growth factor receptor 2 (HER2), fms-like tyrosine kinase 3 (FLT3), Janus kinase 2 (JAK2), mesenchymal-epithelial transition factor (MET), and anaplastic lymphoma kinase (ALK). In embodiments, the cancer is refractory to, or has relapsed after, treatment with a PKI targeted against one or more of EGFR, HER2, and FLT3. In embodiments, the cancer is refractory to, or has relapsed after, treatment with a PKI targeted against one or more of BCR-ABL, B-RAF, JAK2, MET, and ALK.

In embodiments, the cancer is refractory to, or has relapsed after, treatment with a PKI targeted against FLT3. In embodiments, the cancer is refractory to, or has relapsed after, treatment with a PKI targeted against EGFR or HER2. In embodiments, the cancer is refractory to, or has relapsed after, treatment with a therapeutic agent selected from the group consisting of erlotinib, afatinib, lapatinib, dacomitinib, gefitinib, AP32788, poziotinib, osimertinib and EGF816. In embodiments, the cancer is refractory to, or as relapsed after, treatment with a therapeutic agent selected from the group consisting of gilteritinib, tandutinib, crenolanib, sorafenib, midostaurin, and quizartinib. In embodiments, the cancer is acute myeloid leukemia (AML) characterized by one or more activating mutations in FLT3. In embodiments, the one or more activating mutations in FLT3 is selected from the FLT3 internal tandem duplication (ITD) mutation in exon 14 or exon 15, the point mutation at FLT3 D835, the point mutation at 1836, the point mutation FLT3 N676K, and the point mutation F691L in the gatekeeper residue. In embodiments, the one or more activating mutations in FLT3 is the FLT3 ITD mutation. In embodiments, the AML is refractory to or has relapsed after treatment with one or more of cytarabine, daunorubicin, and midostaurin. Additional embodiments related to AML are described infra.

In embodiments, the cancer is refractory to, or has relapsed after, treatment with 5′ azacytidine or decitabine. In embodiments, the cancer is refractory to, or has relapsed after, treatment with cytarabine alone or cytarabine in combination with an anthracycline.

In embodiments, the subject in need of treatment is a subject whose cancer is characterized as having one or more activating mutations in a protein kinase selected from EGFR and HER2. In embodiments, a cancer treated by the methods described herein is characterized by overexpression of EGFR or HER2. In embodiments, the cancer is a non-small cell lung cancer (NSCLC) characterized by one or more EGFR ins20 mutations, or one or more HER2 ins20 mutations, or both.

In embodiments, the one or more activating mutations in EGFR is selected from the group consisting of L858R which may or may not contain the gatekeeper mutation T790M. In embodiments, the EGFR mutation is selected from an exon 20 insertion mutation (ins20). In embodiments, the EGFR ins20 mutation is selected from one or more of E746_A750del, D761_E762insEAFQ, A763_Y764insFQEA, Y764_V765insHH, M766_A767insAI, A767_V769dupASV, A767_S768insTLA, S768_D770dupSVD, S768_V769insVAS, S768_V769insAWT, V769_D770insASV, V769_D770insGV, V769_D770insCV, V769_D770insDNV, V769_D770insGSV, V769_D770insGVV, V769_D770insMASVD, D770_N771insSVD, D770_N771insNPG, D770_N771insAPW, D770_N771insD, D770_N771insDG, D770_N771insG, D770_N771insGL, D770_N771insN, D770_N771insDPH, D770_N771insSVP, D770_N771insSVG, D770_N771insMATP, delN770insGY, N771_PinsH, N771_P772insN, A771_H773dupNPH, delN771insGW, delN771insGF, P772_H773insPR, P772_H773insYNP, P772_H773insX, P772_H773insDPH, P772_H773insDNP, P772_H773insGV, P772_H773insN, P772_H773insV, H773_V774insNPH, H773_V774insH, H773_V774insPH, H773_V774insGNPH, H773_V774insdupHV, H773_V774insG, H773_V774insGH, and V774_C775insHV.

In embodiments, the one or more activating mutations in HER2 is selected from an ins20 mutation. In embodiments, the HER2 ins20 mutation is selected from A775_G776insYVMA, G776>VC, G776_V777insCG, and P781_Y782insGSP.

In embodiments, the subject is one having a refractory or relapsed cancer selected from the group consisting of gastric cancer, colon cancer, prostate cancer, small-cell lung cancer, non-small cell lung cancer (NSCLC), ovarian cancer, lymphoma, acute myeloid leukemia (AML), chronic lymphocytic leukemia (CLL), multiple myeloma, renal cell carcinoma, gastrointestinal stromal tumor, chronic myeloid leukemia, glioblastoma multiforme, astrocytomas, medulloblastomas, melanoma, breast cancer, and pancreatic cancer.

In embodiments, the subject is one having a refractory or relapsed cancer selected from the group consisting of acute granulocytic leukemia, acute lymphocytic leukemia, acute myelogenous leukemia (AML), adrenal cortex carcinoma, adrenal tumor, appendiceal cancer, B-cell lymphoma, bladder carcinoma, brain cancer, breast carcinoma, cervical carcinoma, cervical hyperplasia, choriocarcinoma, chronic granulocytic leukemia, chronic lymphocytic leukemia (CLL), chronic myelogenous leukemia (CML), colorectal carcinoma, endometrial carcinoma, esophageal carcinoma, essential thrombocytosis, gallbladder cancer, gastric cancer, gastrointestinal cancer, genitourinary carcinoma, glioma, hairy cell leukemia, head or neck carcinoma, hepatocellular carcinoma, Hodgkin's lymphoma, Kaposi's sarcoma, leukemia, lung carcinoma, malignant carcinoid carcinoma, malignant hypercalcemia, malignant melanoma, malignant pancreatic insulinoma, mantle cell lymphoma, mesothelioma, multiple myeloma, mycosis fungoides, myeloproliferative neoplasms, neuroblastoma, neuroendocrine tumors, non-Hodgkin's lymphoma, non-small cell lung carcinoma (NSCLC), osteogenic sarcoma, ovarian cancer, ovarian carcinoma, pancreatic carcinoma, penile cancer, pituitary tumor, polycythemia vera, primary macroglobulinemia, primary myelofibrosis, prostatic carcinoma, renal cell carcinoma, rhabdomyosarcoma, sarcoma, skin cancer, small-cell lung carcinoma, soft-tissue sarcoma, stomach carcinoma, T-cell lymphoma, testicular cancer, testicular carcinoma, thyroid carcinoma, thyroid tumor, and Wilms' tumor.

In accordance with the methods described herein, a “subject” includes a mammal. The mammal can be e.g., any mammal, e.g., a human, primate, mouse, rat, dog, cat, cow, horse, goat, camel, sheep or a pig. Preferably, the subject is a human. The term “patient” refers to a human subject.

### Combination Therapy

The present disclosure also provides methods comprising combination therapy. As used herein, “combination therapy” or “co-therapy” includes the administration of a therapeutically effective amount of MPC-0767, or a pharmaceutically acceptable salt thereof, with at least one additional active agent, also referred to herein as an “active pharmaceutical ingredient” (“API”), as part of a treatment regimen intended to provide a beneficial effect from the co-action of the MPC-0767 and the additional active agent. In accordance with the embodiments described below, “the additional API” is understood to refer to the at least one additional API administered in a combination therapy regimen with MPC-0767. In addition, it is understood that more than one of the additional APIs described below may be utilized in the regimen. The terms “combination therapy” or “combination therapy regimen” are not intended to encompass the administration of two or more therapeutic compounds as part of separate monotherapy regimens that incidentally and arbitrarily result in a beneficial effect that was not intended or predicted.

Preferably, the administration of a composition comprising MPC-0767 in combination with one or more additional APIs as discussed herein provides a synergistic response in the subject being treated. In this context, the term “synergistic” refers to the efficacy of the combination being more effective than the additive effects of either single therapy alone.

A synergistic effect is exemplified by the combination of MPC-0767 and venetoclax both against tumor cell lines in vitro and in a systemic survival xenograft study, as discussed in more detail below. Other examples include the synergistic activity of MPC-0767 in combination with 5′ azacytidine, arsenic trioxide (ATO), cytarabine, anthracyclines (e.g. daunorubicin), FLT3 tyrosine kinase inhibitors (e.g., crenolanib and gilterinib), EZH2 inhibitors and Ras/RAF/MEK/ERK pathway inhibitors (e.g., trametinib), for example as shown in Table 1 of Example 10 below (daunorubicin, cytarabine, crenolanib, sorafenib, gilterinib, and venetoclax), in Example 15 (arsenic trioxide), Example 17 (5′ azacytidine), and Example 20 (trametinib).

The synergistic effect of a combination therapy according to the disclosure can permit the use of lower dosages and/or less frequent administration of at least one agent in the combination compared to its dose and/or frequency outside of the combination. Additional beneficial effects of the combination can be manifested in the avoidance or reduction of adverse or unwanted side effects associated with the use of either therapy in the combination alone (also referred to as monotherapy).

In the context of combination therapy, administration of the MPC-0767 composition may be simultaneous with or sequential to the administration of the one or more additional active agents or APIs. In another embodiment, administration of the different components of a combination therapy may be at different frequencies.

In some aspects, the combination therapy encompasses administration of the MPC-0767 composition in combination with a therapeutic agent that enhances the anti-tumor cytotoxic activity of the patient's endogenous immune system. Such agents may act, for example, by enhancing the anti-tumor activity of natural killer cells and/or cytotoxic T cells. Without being bound by any particular theory, the data presented infra indicate that MPC-0767 reduces cell surface PD-L1 expression in both cancer cell lines and in primary cancer cells, leading to increased T cell activation against the cancer cells. Additionally, MPC-0767 treatment sensitizes cancer cells to T cell-mediated cytotoxicity. Accordingly, in embodiments the disclosure provides methods for treating cancer by administering the MPC-0767 composition in combination with a therapeutic agent that enhances anti-tumor immunity, for example an inhibitor of a checkpoint signaling pathway involving a programmed death 1 (PD-1) receptor and/or its ligands (PD-L1/2) and may include therapeutic antibodies or fragments thereof with multiple specificities that engage T cells or natural killer cells. In embodiments, these may include bispecific antibodies, BiTE (bispecific T cell engager), scBsTaFv (single-chain bispecific tandem fragment variable), bsscFv (bispecific single-chain Fv), BiKE (bispecific killer-cell engager), DART (Dual-Affinity Re-Targeting), TandAb (Tandem Diabodies) sctb (Single-chain Fv Triplebody) BIf (bispecific scFv Immunofusion), and DVD-Ig (DualVariable-Domain Immunoglobulin).

In embodiments, the disclosure provides methods for treating a hematologic cancer by administering the MPC-0767 composition in combination with a therapeutic agent that enhances anti-tumor immunity, for example a bispecific therapeutic antibody or fragment thereof against CD3 and CD19 (Blincyto, MGD011), CD3 and BCMA (EM801), or CD3 and CD20 (REGN1979). In embodiments where the cancer is AML, the bispecific therapeutic antibody or fragment thereof may encompass one that targets CD3 and CD33 (AMG-330, AMG-673, AMV-654), CD3 and CD123 (MGD006/580880, JNJ-63709178), CD3 and CLL-1, or CD3 and WT1. In the context of solid tumors, including non-small cell lung cancer (NSCLC) and breast cancer, the bispecific therapeutic antibody or fragment thereof may encompass one that targets CD3 and EGFR (EGFRBi-aATC), CD3 and HER2 (ertumaxomab), or CD3 and EpCAM (Catumaxomab, MT110/AMG 110/Solitomab).

In embodiments, the additional API may be formulated for co-administration with an MPC-0767 composition in a single dosage form. The additional API(s) may also be administered separately from the dosage form that comprises the MPC-0767. When the additional active agent is administered separately from MPC-0767, it can be by the same or a different route of administration, and/or at the same or different time.

In embodiments, the additional API for use in combination therapy with MPC-0767 is selected from a chemotherapeutic agent, a protein kinase inhibitor (PKI), an FLT3 inhibitor, a PD-1/PD-L1 inhibitor, a CTLA-4 inhibitor, a Bcl-2 pathway inhibitor, a Ras/Raf/MEK/ERK pathway inhibitor, an EZH2 inhibitor, arsenic trioxide (ATO), and a DNA methyltransferase inhibitor (DNMT).

In embodiments, the chemotherapeutic agent is a platinum based anti-neoplastic agent, a topoisomerase inhibitor, a nucleoside metabolic inhibitor, an alkylating agent, an intercalating agent, a tubulin binding agent, an inhibitor of DNA repair, and combinations thereof. In embodiments, the chemotherapeutic agent is selected from docetaxel, carboplatin, cisplatin, and pemetrexed.

In embodiments, the PKI is an EGFR or HER2 targeted PKI. In embodiments the PKI is selected from erlotinib, afatinib, lapatinib, dacomitinib, gefitinib, AP32788, poziotinib, osimertinib, and EGF816, and combinations thereof.

In embodiments, the FLT3 inhibitor is selected from crenolanib, tandutinib, gilteritinib, midostaurin, quizartinib, and sorafenib.

In embodiments, the PD-1/PD-L1 inhibitor is an agent that inhibits the signaling of PD-1 and its ligands PD-L1/2 and is selected from AMP-224, AMP-514/MEDI-0680, atezolizumab (Tenectriq®, MPDL3280A), avelumab (MSB0010718C), BGB-A317, BMS936559, cemiplimab (REGN2810), durvalumab (MEDI-4736), JTX-4014, nivolumab (Opdivo®, BMS-936558), pembrolizumab (Keytruda®, MK-3475), and SHR-1210.

In embodiments, the CTLA-4 inhibitor is Ipilimumab (Yervoy®).

In embodiments, the Bcl-2 pathway inhibitor is selected from ABT-737, AT-101 (Gossypol), APG-1252, A1155463, A1210477, navitoclax, obatoclax, sabutoclax, venetoclax, S 55746, and WEHI-539. In embodiments, the Bcl-2 pathway inhibitor is an inhibitor of BCL2, BCLXL, or MCL1. In embodiments, the Bcl-2 pathway inhibitor is selected from AMG-176, MIK665 and 5641315. In embodiments, the Bcl-2 pathway inhibitor is selected from ABT-737, navitoclax, and venetoclax. In embodiments, the Bcl-2 pathway inhibitor is venetoclax. In embodiments, the Bcl-2 pathway inhibitor is selected from TW-37 (Wang et al., J Med Chem. 2006 Oct. 19; 49(21):6139-42) and HA14-1 (Wang et al., Proc Natl Acad Sci USA. 2000 Jun. 20; 97(13):7124-9).

In embodiments, the Ras/Raf/MEK/ERK pathway inhibitor is selected from a Raf inhibitor such as vemurafenib, sorafenib, or dabrafenib, a MEK inhibitor such as AZD6244 (Selumetinib), PD0325901, GSK1120212 (Trametinib), U0126-EtOH, PD184352, RDEA119 (Rafametinib), PD98059, BIX 02189, MEK162 (Binimetinib), AS-703026 (Pimasertib), SL-327, BIX02188, AZD8330, TAK-733, cobimetinib or PD318088, and an ERK inhibitor such as LY3214996, BVD-523 or GDC-0994.

In embodiments, the EZH2 inhibitor is selected from EPZ6438, CPI-1205, GSK343, GSK2816126, MAK-683 and PF-06821497.

In embodiments, the additional API for use in combination therapy with MPC-0767 is arsenic trioxide (ATO).

In embodiments, the DNA methyltransferase inhibitor (DNMT) is 5′ azacytidine.

In embodiments, the additional API for use in combination therapy with MPC-0767 is selected from a CTLA-4 inhibitor, an HDAC inhibitor, an ImiD, a VEGF inhibitor, such as an anti-VEGFR antibody, an mTOR inhibitor such as everolimus or temsirolimus, a DNA methylation inhibitor, a steroid hormone agonist or antagonist, a metabolic enzyme inhibitor, a proteasome inhibitor, an anti-CD20 antibody, an adenosine receptor 2A antagonist, a toll-receptor agonist or antagonist, and an immunostimulatory cytokine.

In embodiments, the additional API for use in combination therapy with MPC-0767 is selected from daunorubicin, doxorubicin, epirubicin, mitoxantrone, idarubicin, and cytarabine, and combinations thereof. In embodiments, the additional API is selected from crenolanib, cytarabine, daunorubicin, gilteritinib, sorafenib, and venetoclax. In embodiments, the additional API is venetoclax.

In embodiments, the additional API for use in combination therapy with MPC-0767 is selected from an inhibitor of the mTOR pathway, a PI3K inhibitor, a dual PI3K/mTOR inhibitor, a SRC inhibitor, a VEGF inhibitor, a Janus kinase (JAK) inhibitor, a Raf inhibitor, an Erk inhibitor, a Ras/Raf/MEK/ERK pathway inhibitor, an Akt inhibitor, a farnesyltransferase inhibitor, a c-MET inhibitor, a histone-modulating inhibitor, an anti-mitotic agent, a tyrosine kinase inhibitor (TKI) inhibitor, a polyether antibiotic, a CTLA-4 inhibitor, a multi-drug resistance efflux inhibitor, a multi-drug resistance efflux inhibitor, and a therapeutic cytokine, such as interleukin-2 (IL-2).

In embodiments, the mTOR inhibitor is selected from the group consisting of rapamycin (also referred to as sirolimus), everolimus, temsirolimus, ridaforolimus, umirolimus, zotarolimus, AZD8055, INK128, WYE-132, Torin-1, pyrazolopyrimidine analogs PP242, PP30, PP487, PP121, KU0063794, KU-BMCL-200908069-1, Wyeth-BMCL-200910075-9b, INK-128, XL388, AZD8055, P2281, and P529. See, e.g., Liu et al. Drug Disc. Today Ther. Strateg., 6(2): 47-55 (2009).

In embodiments, the mTOR inhibitor is trans-4-[4-amino-5-(7-methoxy-1H-indol-2-yl)imidazo[5,1-f][1,2,4]triazin-7-yl]cyclohexane carboxylic acid (also known as OSI-027), and any salts, solvates, hydrates, and other physical forms, crystalline or amorphous, thereof. See US 2007/0112005. OSI-027 can be prepared according to US 2007/0112005, incorporated herein by reference. In one embodiment, the mTOR inhibitor is OXA-01. See e.g., WO 2013152342 A1.

In embodiments, the PI3K inhibitor is selected from the group consisting of GS-1101 (Idelalisib), GDC0941 (Pictilisib), LY294002, BKM120 (Buparlisib), PI-103, TGX-221, IC-87114, XL 147, ZSTK474, BYL719, AS-605240, PIK-75, 3-methyladenine, A66, PIK-93, PIK-90, AZD6482, IPI-145 (Duvelisib), TG100-115, AS-252424, PIK294, AS-604850, GSK2636771, BAY 80-6946 (Copanlisib), CH5132799, CAY10505, PIK-293, TG100713, CZC24832 and HS-173.

In embodiments, the dual PI3K/mTOR inhibitor is selected from the group consisting of, GDC-094, WAY-001, WYE-354, WAY-600, WYE-687, Wyeth-BMCL-200910075-16b, Wyeth-BMCL-200910096-27, KU0063794 and KUBMCL-200908069-5, NVP-BEZ235, XL-765, PF-04691502, GDC-0980 (Apitolisib), GSK1059615, PF-05212384, BGT226, PKI-402, VS-558 and GSK2126458. See, e.g., Liu et al. Drug Disc. Today Ther. Strateg., 6(2): 47-55 (2009), incorporated herein by reference.

In embodiments, the mTOR pathway inhibitor is a polypeptide (e.g., an antibody or fragment thereof) or a nucleic acid (e.g., a double-stranded small interfering RNA, a short hairpin RNA, a micro-RNA, an antisense oligonucleotide, a locked nucleic acid, or an aptamer) that binds to and inhibits the expression level or activity or a protein (or nucleic acid encoding the protein) in the mTOR pathway. For example, the polypeptide or nucleic acid inhibits mTOR Complex 1 (mTORC1), regulatory-associated protein of mTOR (Raptor), mammalian lethal with SEC13 protein 8 (MLST8), proline-rich Akt substrate of 40 kDa (PRAS40), DEP domain-containing mTOR-interacting protein (DEPTOR), mTOR Complex 2 (mTORC2), rapamycin-insensitive companion of mTOR (RICTOR), G protein beta subunit-like (GβL), mammalian stress-activated protein kinase interacting protein 1 (mSIN1), paxillin, RhoA, Ras-related C3 botulinum toxin substrate 1 (Rac1), Cell division control protein 42 homolog (Cdc42), protein kinase C α (PKCα), the serine/threonine protein kinase Akt, phosphoinositide 3-kinase (PI3K), p70S6K, Ras, and/or eukaryotic translation initiation factor 4E (eIF4E)-binding proteins (4EBPs), or the nucleic acid encoding one of these proteins.

In embodiments, the SRC inhibitor is selected from the group consisting of bosutinib, saracatinib, dasatinib, ponatinib, KX2-391, XL-228, TG100435/TG100855, and DCC2036. See, e.g., Puls et al. Oncologist. 2011 May; 16(5): 566-578. In one embodiment, the SRC inhibitor is a polypeptide (e.g., an antibody or fragment thereof) or nucleic acid (e.g., a double-stranded small interfering RNA, a short hairpin RNA, a micro-RNA, an antisense oligonucleotide, a locked nucleic acid, or an aptamer) that binds to and inhibits the expression level or activity of the SRC protein or a nucleic acid encoding the SRC protein.

In embodiments, the VEGF inhibitor is selected from axitinib, bevacizumab, cabozantinb, lenvatinib, motesanib, pazopanib, regorafenib, sorafenib, and sunitinib. In embodiments, the VEGF inhibitor is a polypeptide (e.g., an antibody or fragment thereof) or nucleic acid (e.g., a double-stranded small interfering RNA, a short hairpin RNA, a micro-RNA, an antisense oligonucleotide, a morpholino, a locked nucleic acid, or an aptamer) that binds to and inhibits the expression level or activity of a VEGF protein, a VEGF receptor protein, or a nucleic acid encoding one of these proteins. For example, the VEGF inhibitor is a soluble VEGF receptor (e.g., a soluble VEGF-C/D receptor (sVEGFR-3)).

In embodiments, the JAK inhibitor is selected from facitinib, ruxolitinib, baricitinib, CYT387 (CAS number 1056634-68-4), lestaurtinib, pacritinib, and TG101348 (CAS number 936091-26-8). In one embodiment, the JAK inhibitor is a polypeptide (e.g., an antibody or fragment thereof) or nucleic acid (e.g., a double-stranded small interfering RNA, a short hairpin RNA, a micro-RNA, an antisense oligonucleotide, a morpholino, a locked nucleic acid, or an aptamer) that binds to and inhibits the expression level or activity of a JAK (e.g., JAK1, JAK2, JAK3, or TYK2) or a nucleic acid encoding the JAK protein.

In embodiments, the Raf inhibitor is selected from PLX4032 (vemurafenib), sorafenib, PLX-4720, GSK2118436 (dabrafenib), GDC-0879, RAF265, AZ 628, NVP-BHG712, SB90885, ZM 336372, GW5074, TAK-632, CEP-32496 and LGX818 (Encorafenib). In embodiments, the Raf inhibitor is a polypeptide (e.g., an antibody or fragment thereof) or nucleic acid (e.g., a double-stranded small interfering RNA, a short hairpin RNA, a micro-RNA, an antisense oligonucleotide, a morpholino, a locked nucleic acid, or an aptamer) that binds to and inhibits the expression level or activity of a Raf (e.g., A-Raf, B-Raf, C-Raf) or a nucleic acid encoding the Raf protein.

In embodiments, the ERK inhibitor is selected from LY3214996, BVD-523 and GDC-0994.

In embodiments, the Ras/Raf/MEK/ERK pathway inhibitor is a Raf inhibitor or an Erk inhibitor, as described above. In embodiments, the Ras/Raf/MEK/ERK pathway inhibitor is a MEK inhibitor selected from AZD6244 (Selumetinib), PD0325901, GSK1120212 (Trametinib), U0126-EtOH, PD184352, RDEA119 (Rafametinib), PD98059, BIX 02189, MEK162 (Binimetinib), AS-703026 (Pimasertib), SL-327, BIX02188, AZD8330, TAK-733, cobimetinib and PD318088. In embodiments, the MEK inhibitor is a polypeptide (e.g., an antibody or fragment thereof) or nucleic acid (e.g., a double-stranded small interfering RNA, a short hairpin RNA, a micro-RNA, an antisense oligonucleotide, a morpholino, a locked nucleic acid, or an aptamer) that binds to and inhibits the expression level or activity of a MEK (e.g., MEK-1, MEK-2) or a nucleic acid encoding the MEK protein.

In embodiments, the Akt inhibitor is selected from MK-2206, KRX-0401 (perifosine), GSK690693, GDC-0068 (Ipatasertib), AZD5363, CCT128930, A-674563, PHT-427. In embodiments, the Akt inhibitor is a polypeptide (e.g., an antibody or fragment thereof) or nucleic acid (e.g., a double-stranded small interfering RNA, a short hairpin RNA, a micro-RNA, an antisense oligonucleotide, a morpholino, a locked nucleic acid, or an aptamer) that binds to and inhibits the expression level or activity of an Akt (e.g., Akt-1, Akt-2, Akt-3) or a nucleic acid encoding an Akt protein.

In embodiments, the farnesyltransferase inhibitor is selected from LB42708 or tipifarnib. In one embodiment, the farnesyltransferase inhibitor is a polypeptide (e.g., an antibody or fragment thereof) or nucleic acid (e.g., a double-stranded small interfering RNA, a short hairpin RNA, a micro-RNA, an antisense oligonucleotide, a morpholino, a locked nucleic acid, or an aptamer) that binds to and inhibits the expression level or activity of farnesyltransferase or a nucleic acid encoding the farnesyltransferase protein.

In embodiments, the c-MET inhibitor is selected from crizotinib, tivantinib, cabozantinib, foretinib. In one embodiment, the c-MET inhibitor is a polypeptide (e.g., an antibody or fragment thereof, exemplified by onartuzumab) or nucleic acid (e.g., a double-stranded small interfering RNA, a short hairpin RNA, a micro-RNA, an antisense oligonucleotide, a morpholino, a locked nucleic acid, or an aptamer) that binds to and inhibits the expression level or activity of c-MET or a nucleic acid encoding the c-MET protein or the HGF ligand, such as ficlatuzumab or rilotumumab.

In embodiments, the histone-modulating inhibitor is selected from anacardic acid, C646, MG149 (histone acetyltransferase), GSK J4 Hcl (histone demethylase), MAK-683 (PRC2 inhibitor), BIX 01294 (histone methyltransferase), MK0683 (Vorinostat), MS275 (Entinostat), LBH589 (Panobinostat), Trichostatin A, MGCD0103 (Mocetinostat), Tasquinimod, TMP269, Nexturastat A, RG2833, and PDX101 (Belinostat). In embodiments, the histone-modulating inhibitor is an EZH2 inhibitor selected from GSK343, EPZ6438 (Tazemetostat), CPI-1205, GSK2816126, and PF-06821497.

In embodiments, the anti-mitotic agent is selected from Griseofulvin, vinorelbine tartrate, paclitaxel, docetaxel, vincristine, vinblastine, Epothilone A, Epothilone B, ABT-751, CYT997 (Lexibulin), vinflunine tartrate, Fosbretabulin, GSK461364, ON-01910 (Rigosertib), Ro3280, BI2536, NMS-P937, BI 6727 (Volasertib), HMN-214 and MLN0905.

In embodiments, the tyrosine kinase inhibitor (TKI) is selected from Votrient, Axitinib, Bortezomib, Bosutinib, Carfilzomib, Crizotinib, Dabrafenib, Dasatinib, Erlotinib, Gefitinib, Ibrutinib, Imatinib, Lapatinib, Nilotinib, Pegaptanib, Ponatinib, Regorafenib, Ruxolitinib, Sorafenib, Sunitinib, Trametinib, Vandetanib, Vemurafenib, and Vismodegib.

In one embodiment, the polyether antibiotic is selected from sodium monensin, nigericin, valinomycin, salinomycin.

In embodiments, the CTLA-4 inhibitor is selected from tremlimumab and ipilimumab.

In embodiments, the at least one additional API(s) is a checkpoint inhibitor. Treatment with these compounds works by targeting molecules that serve as checks and balances on immune responses. By blocking these inhibitory molecules or, alternatively, activating stimulatory molecules, these treatments are designed to unleash or enhance pre-existing anti-cancer immune responses. In embodiments, the checkpoint inhibitor may be selected from an antibody such as an anti-CD27 antibody, an anti-B7-H3 antibody, an anti-KIR antibody, an anti-LAG-3 antibody, an anti-4-1BB/CD137 antibody, an anti-GITR antibody (e.g., TRX518, MK-4166), pembrolizumab (Keytruda™, a PD-1 antibody), MPDL3280A (a PD-L1 antibody), varlilumab (CDX-1127, an anti-CD27 antibody), MGA217 (an antibody that targets B7-H3), lirilumab (a KIR antibody), BMS-986016 (a LAG-3 antibody), urelumab (a 4-1BB/CD137 antibody), an anti-TIM3 antibody, MEDI-0562 (a OX40 antibody), SEA-CD40 (an anti-CD40 antibody), tremelimumab (anti-CTLA4 antibody), an anti-OX40 antibody, and an anti-CD73 antibody. In embodiments, the checkpoint inhibitor is selected from a small molecule inhibitor of CD73 (as described, for example, in Cancer Immunol Res 2016;4 (11 Suppl):Abstract nr PR10). In embodiments, the checkpoint inhibitor is selected from varlilumab, MGA217, lirilumab, BMS-986016, urelumab, MEDI-0562, SEA-CD40, TRX518, or MK-4166.

In embodiments, the additional API is a DNA repair inhibitor selected from olaparib, rucaparib, niraparib, talazoparib veliparib, CEP-9722, and CEP-8983.

In embodiments, additional API(s) is selected from ddAC, panobinostat, exemestane, letrozole, esartinib, merestinib, mocetinostat, etinostat, motolimod, ibrutinib, lenalidomide, idelalisib, enzalutamide, prednisone, dexamethasone, vinflunine, vorinostat, galunisertib, bendamustine, oxaliplatin, leucovorin, guadecitabine, trametinib, vemurafenib, dacarbazine, apatinib, pomalidomide, carfilzomib, sorafenib, 5-fluorouracil, CB-839, CB-1158, GDC-0919, LXH254, AZD4635, AZD9150, PLX3397, LCL161, PBF-509, Sym004, trastuzumab, obinutuzumab, B-701, utomilumab, rituximab, NKTR-214, PEGlnterferon 2A, RO7009789, MEDI9447, MK-1248, LY2510924, ARRY-382, MEDI0562, LAG525, NIS793, GWN323, JTX-2011, TSR-022, and REGN3767.

In embodiments, the additional API is directed towards targeted therapy, wherein the treatment targets the cancer's specific genes, proteins, or the tissue environment that contributes to cancer growth and survival. This type of treatment blocks the growth and spread of cancer cells while limiting damage to healthy cells. In embodiments, the at least one additional API is directed towards anti-angiogenesis therapy, wherein the treatment focuses on stopping angiogenesis, which is the process of making new blood vessels. Because a tumor needs the nutrients delivered by blood vessels to grow and spread, the goal of anti-angiogenesis therapies is to “starve” the tumor. One anti-angiogenic drug, bevacizumab (Avastin), has been shown to slow tumor growth for people with metastatic renal carcinoma. Bevacizumab combined with interferon slows tumor growth and spread.

In embodiments, the additional API is directed towards immunotherapy, also called biologic therapy, which is designed to boost the body's natural defenses to fight cancer. It uses materials made either by the body or in a laboratory to improve, target, or restore immune system function. For example, interleukin-2 (IL-2) is a drug that has been used to treat kidney cancer as well as AM0010, and interleukin-15. They are cellular hormones called cytokines produced by white blood cells and are important in immune system function, including the destruction of tumor cells. Alpha-interferon is another type of immunotherapy used to treat kidney cancer that has spread. Interferon appears to change the proteins on the surface of cancer cells and slow their growth. Many combination therapies of IL-2 and alpha-interferon for patients with advanced kidney cancer combined with chemotherapy are more effective than IL-2 or interferon alone.

In embodiments, the additional API is a cancer vaccine, designed to elicit an immune response against tumor-specific or tumor-associated antigens, encouraging the immune system to attack cancer cells bearing these antigens. In embodiments, the cancer vaccine is AGS-003, DCVax, NY-ESO-1 or a personalized vaccine derived from patient's cancer cells.

In embodiments, the additional API is an immunostimulant, such as a recombinant protein, used to activate the immune system to attack cancer cells. In embodiments, the immunostimulant is denenicokin (recombinant IL-21).

In embodiments, the additional API is a small molecule that modulates the immune system to encourage the elimination of cancer cells. In embodiments, the small molecule is epacadostat or navoximod (both IDO inhibitors), or PLX3397 (an inhibitor of CSF-1R).

In embodiments, the additional API may be the patient's own immune cells which have been removed from a patient, genetically modified or treated with chemicals to enhance their activity, and then re-introduced into the patient with the goal of improving the immune system's anti-cancer response.

“Combination therapy” also embraces the administration of MPC-0767 in further combination with non-drug therapies (e.g., surgery or radiation treatment). Where the combination therapy further comprises a non-drug treatment, the non-drug treatment may be conducted at any suitable time so long as a beneficial effect from the co-action of the combination of the therapeutic compounds and non-drug treatment is achieved. For example, in appropriate cases, the beneficial effect is still achieved when the non-drug treatment is temporally removed from the administration of the therapeutic compounds, perhaps by days or even weeks.

The non-drug treatment can be selected from chemotherapy, radiation therapy, hormonal therapy, anti-estrogen therapy, gene therapy, surgery (e.g. radical nephrectomy, partial nephrectomy, laparoscopic and robotic surgery), radiofrequency ablation, and cryoablation. For example, a non-drug therapy is the removal of an ovary (e.g., to reduce the level of estrogen in the body), thoracentesis (e.g., to remove fluid from the chest), paracentesis (e.g., to remove fluid from the abdomen), surgery to remove or shrink angiomyolipomas, lung transplantation (and optionally with an antibiotic to prevent infection due to transplantation), or oxygen therapy (e.g., through a nasal cannula containing two small plastic tubes or prongs that are placed in both nostrils, through a face mask that fits over the nose and mouth, or through a small tube inserted into the windpipe through the front of the neck, also called transtracheal oxygen therapy).

### Biomarker Assays for Diagnosis and Treatment

In embodiments, the disclosure provides biomarkers that can be used to predict the sensitivity of a cancer to treatment with an HSP90 inhibitor, and in particular sensitivity to MPC-0767. In this context, ‘sensitivity’ refers to response to therapy, or therapeutic responsiveness associated with treating the cancer, for example as described in the section below entitled “Treating Cancer.” The terms ‘responsiveness’ in the context of response to an anti-cancer therapy such as MPC-0767, and ‘sensitivity’ in the context of sensitivity to treatment with an anti-cancer therapy such as MPC-0767, are used interchangeably herein.

In embodiments, the disclosure provides methods for treating a cancer or predicting the responsiveness of a cancer to treatment with an HSP90 inhibitor, and in particular sensitivity to MPC-0767, the methods comprising determining or receiving the status of one or more biomarkers of MPC-0767 resistance or sensitivity. For example, as disclosed herein, AML cancer cells harboring activating mutations in FLT3, and particularly FLT3-ITD mutations, are highly sensitive to the cytotoxic activity of MPC-0767. Accordingly, the disclosure provides methods for treating AML and methods for predicting responsiveness to treatment with an HSP90 inhibitor, and in particular sensitivity to MPC-0767, the methods comprising determining or receiving the FLT3 status of the AML.

In further embodiments, the one or more biomarkers of MPC-0767 resistance or sensitivity is an activating mutation in NRAS or KRAS in AML cells having a normal or wild-type FLT3 status. In this context, the terms ‘normal’ and ‘wild-type’ are used interchangeably to refer to the wild type allele of the gene which produces a protein having normal activity. As described herein, an activating mutation in NRAS or KRAS in AML cells having a normal FLT3 status indicates that the cancer cells are likely to be resistant to treatment with MPC-0767 but are likely to be responsive to treatment with a combination therapy comprising MPC-0767 and a Ras/Raf/MEK/ERK pathway inhibitor.

In further embodiments, the one or more biomarkers of MPC-0767 resistance or sensitivity is an FLT3-ITD mutation or an FLT3 tyrosine kinase domain (FLT3-TKD) mutation.

In further embodiments, the one or more biomarkers of MPC-0767 resistance or sensitivity is KDM6A or EZH2. As described herein, a loss of function mutation in KDM6A indicates that the cancer cells are likely to be resistant to treatment with MPC-0767 but are likely to be responsive to treatment with a combination therapy comprising MPC-0767 and an EZH2 inhibitor. In embodiments, an EZH2 loss of function mutation is predicted to result in a cancer that is responsive to MPC-0767 monotherapy and an EZH2 gain of function mutation is predicted to result in a cancer that is resistant to MPC-0767 monotherapy.

The disclosure provides biomarkers that indicate high sensitivity of cancer cells to the cytotoxic effects of MPC-0767. In embodiments, the disclosure provides genetic biomarkers in the form of one or more variants in a polynucleotide sequence encoding a gene, for example FLT3, NRAS, KRAS, KDM6A, and EZH2. In embodiments, the polynucleotide variant may result in an amino acid change in the encoded protein. In embodiments, the biomarker is a marker of gene expression, for example mRNA or protein abundance, e.g., expression levels of KRAS or NRAS.

In embodiments, the one or more activating mutations in NRAS or KRAS is a mutation in the polynucleotide sequence encoding the Ras protein that results in an amino acid change selected from the group consisting of A146T and G13D of KRAS; or Q61L, Q61H, and G12D of NRAS. In embodiments, the one or more activating mutations in KRAS is selected from KRAS G12(V,C,S,R,D,N,A), G13(D,C), Q22K, Q61(H,L,R), and K117NA146(T/V) where the letter designations refer to the one-letter amino acid symbols recommended by the IUPAC-IUB Biochemical Nomenclature Commission.

In embodiments, the one or more variants is a variant in a polynucleotide sequence of a gene that is part of a molecular signaling or synthetic pathway, for example a Ras/Raf/MEK/ERK pathway, a Bcl-2 pathway or a histone methyltransferase/demethylase pathway.

In embodiments, the methods described here may include determining the presence of one or more of the biomarkers disclosed here in a biological sample of cancer cells from a subject. As noted above, the biomarker may be a genetic biomarker in the form of one or more variants in a polynucleotide sequence, which may result in an amino acid change in the encoded protein. Accordingly, the methods described here may include a step of detecting the one or more variants in a polynucleotide sequence. Where the variant is in an exon of a gene encoding a protein, the variant may be detected either in the genomic DNA or in the RNA of the cancer cells.

In embodiments, the methods may comprise determining the subject's genotype to detect the presence of one or more of the genetic biomarkers. Genotype may be determined by techniques known in the art, for example, PCR-based methods, DNA sequencing, 5′ exonuclease fluorescence assay, sequencing by probe hybridization, dot blotting, and oligonucleotide array hybridization analysis, for example, high-throughput or low density array technologies (also referred to as microarrays and gene chips), and combinations thereof. Other specific techniques may include dynamic allele-specific hybridization, molecular beacons, restriction fragment length polymorphism (RFLP)-based methods, flap endonuclease-based methods, primer extension, 5′-nuclease-based methods, oligonucleotide ligase assays, single-stranded conformation polymorphism assays (SSCP), temperature-gradient gel electrophoresis, denaturing high-performance liquid chromatography (HPLC), high-resolution melting analysis, DNA mismatch-binding methods, capillary electrophoresis, and next-generation sequencing (NGS) methods. Real-time PCR methods that can be used to detect SNPs, include, e.g., Taqman or molecular beacon-based assays (U.S. Pat. Nos. 5,210,015; 5,487,972; and PCT WO 95/13399). Genotyping technology is also commercially available, for example from companies such as Applied Biosystems, Inc (Foster City, Calif.).

In embodiments, genotype may be determined by a method selected from direct manual sequencing, automated fluorescent sequencing, single-stranded conformation polymorphism assays (SSCPs), clamped denaturing gel electrophoresis (CDGE), denaturing gradient gel electrophoresis (DGGE), mobility shift analysis, restriction enzyme analysis, heteroduplex analysis, chemical mismatch cleavage (CMC), and RNase protection assays.

In embodiments, the method of detecting the presence of a biomarker may comprise a step of contacting a set of SNP-specific primers with DNA extracted from a sample of cancer cells from the subject, allowing the primers to bind to the DNA, and amplifying the SNP containing regions of the DNA using a polymerase chain reaction.

In embodiments, the methods described here may comprise receiving, in a computer system, the patient's genotype for one or more of the biomarkers described here. In one embodiment, a user enters the patient's genotype in the computer system. In one embodiment, the patient's genotype is received directly from equipment used in determining the patient's genotype.

In further embodiments, the biomarker may be a marker of gene expression, for example mRNA or protein abundance. Suitable methods for detecting gene expression of a biomarker described here include methods comprising microarray expression analysis, PCR-based methods, in-situ hybridization, Northern immunoblotting and related probe hybridization techniques, single molecule imaging technologies such as nCounter® or next generation sequencing methods such as RNA-seg™ (Life Technologies) and SAGE technologies™ and combinations of the foregoing. In embodiments, the methods may comprise detection of protein expression using a suitable method comprising one or more of immunohistochemisty, mass spectrophotometry, flow cytometry, an enzyme-linked immunoabsorbant assay, Western immunoblotting and related probe hybridization techniques, multiplex immunoassay (e.g., Luminex®, MesoScale™ Discovery, SIMOA™), single molecule imaging technologies such as nCounter®, and aptamer-based multiplex proteomic technologies such as SOMAscan®.

In embodiments, the methods may further comprise obtaining a biological sample of cancer cells from the subject in need of treatment, for example by a biopsy procedure. In this context, a biopsy procedure comprises extracting a sample of cancer cells or tissue comprising cancer cells from the subject. The biopsy may be performed, for example, as an incisional biopsy, a core biopsy, or an aspiration biopsy, e.g., fine needle aspiration.

In embodiments, the methods may further comprise obtaining a biological sample of cancer cells from whole blood.

### Acute Myelogenous Leukemia (AML)

AML is a hematopoietic cancer with significant unmet medical need and limited therapy options. Multiple genetic lesions have been identified which contribute to disease heterogeneity in AML and likely explain the historic difficulty in developing new targeted therapies. See e.g., Cancer Genome Atlas Research Network, NEJM 2013 368: 2059; Grimwade et al., Blood 2016 129:29; Papaemmanuil et al., NEJM 2016; 374: 2209; Breitenbuecher et al., Blood 2009 113:4074; Kindler et al., Blood 2005 105:335. Mutation of the cell surface receptor fms-like tyrosine kinase (FLT3) is found in ˜30% of AML patients, and is associated with a significantly poorer prognosis (Papaemmanuil et al, NEJM 2016; 374: 2209). FLT3 mutations fall into two general categories. The first are point mutations that occur within the activation loop of the tyrosine kinase domain leading to constitutive activation, for example at D835. Specific point mutations that lead to constitutively active FLT3 include mutations at residues F691, D835, N676, 1836, and Y842 (Kindler et al. Blood 2005). The second are the internal tandem duplications (FLT3 ITDs) which occur in or adjacent to the juxtamembrane domain of the receptor. These mutations can vary in size ranging from 3 to more than 400 base pairs. Since they always occur in multiples of 3, the reading frame is maintained. These duplications are usually contained within exon 14, near residues 590-600 of FLT. An ITD has also been observed within the kinase domain (Breitenbuecher et al., Blood 2009). Receptors carrying the FLT3 ITD mutations are constitutively autophosphorylated, and therefore constitutively active. The FLT3 pathway activates downstream kinases involved in cell survival and cell proliferation including JAK2, STAT3, STATS, PI3-K, and AKT. The PKI midostaurin is FDA-approved for treating AML. FLT3 is a client protein of HSP90 and HSP90 stabilizes the FLT3 ITD mutant protein. Higher HSP90 levels are associated with poorer survival of AML patients after induction therapy.

The standard-of-care treatment for AML is a combination of initial induction therapy with cytarabine and an anthracycline, such as daunorubicin, followed by consolidation therapy with additional cytotoxic agents such as cytarabine, mitxantrone, and/or etoposide. See Ramos et al. J. Clin. Med. 2015 6: 665; Pratz and Levis, Blood 2017 129:565. Recently, midostaurin has been approved by the U.S. Food and Drug Administration as a first line therapy in combination with the “standard of care”, cytarabine and anthracycline induction. Additional FLT3 inhibitors are in clinical development (Stone et al. NEJM 2017 377: 454) but as with protein tyrosine kinase inhibitors generally, the development of resistance to FLT3 inhibitors remains a concern. See e.g., Weisberg et al., Oncogene 2010 19: 5120. One key mechanism of drug resistance is acquired mutations in FLT3 that reduce inhibitor binding. For example, a FLT3 ITD patient treated with midostaurin developed resistance due to a mutation at position N676K, within the kinase domain (Heidel et al., Blood. 2006), and the FLT3 D835 and gatekeeper F691L mutations confer resistance to quizartinib and sorafenib. In addition, AML blasts from a patient refractory to crenolanib contained the F691L mutation, and ex-vivo assaying of these blasts confirmed resistance to crenolanib and gilteritinib (Lee et al., Blood 2017). These findings support the notion that the F691L mutation reduces potency of crenolanib and gilteritinib. Another mechanism for developing drug resistance is through the activation of other signaling pathways, such as in response to stromal factors in the cellular microenvironment.

As described in more detail in the examples below, AML cells having FLT3 ITD mutations are unexpectedly sensitive to treatment with MPC-0767, both in vitro and in vivo. Remarkably, AML cells which have developed resistance to other protein tyrosine kinase inhibitors via multiple different mechanisms (e.g., acquisition of mutations in FLT3 and via stromal signaling) also remain sensitive to MPC-0767. In addition, MPC-0767 abrogates interferon gamma induced PD-L1 expression in primary AML cells. Further, MPC-0767 acts synergistically with a number of other active agents used to treat AML, including daunorubicin, venetoclax, cytarabine, crenolanib, gilteritinib, and sorafenib. MPC-0767 also showed a surprising ability to synergize with venetoclax in a systemic xenograft study using FLT3-ITD AML cells and significantly improved animal survival. Taken together, the results presented here support MPC-0767 as an attractive new therapy for treating AML and other cancers, both as monotherapy and in combination with other APIs.

Accordingly, the disclosure provides methods of treating AML in a subject in need thereof by administering to the subject a therapeutically effective amount of MPC-0767. In embodiments, the subject in need is one whose AML is characterized by having one or more activating mutations in FLT3 selected from the FLT3 ITD mutation, FLT3 D835, FLT3 1836, and FLT3 N676K, or at the gatekeeper residue F691. In embodiments, the AML is relapsed/refractory to treatment with a protein kinase inhibitor. In embodiments, the AML is relapsed/refractory to treatment with an FLT3 protein kinase inhibitor. In embodiments, the AML is relapsed/refractory to treatment with one or more of gilteritinib, crenolanib, tandutinib, midostaurin, quizartinib, and sorafenib.

In embodiments, the disclosure also provides methods of combination therapy comprising MPC-0767 in combination with the standard of care treatment for AML. In embodiments, MPC-0767 is administered following initial induction therapy with cytarabine and an anthracycline. In embodiments, MPC-0767 is administered alone following initial induction therapy, or in combination with one or more of midostaurin, quizartinib, gilteritinib, crenolanib, tandutinib, venetoclax, and sorafenib. In embodiments, MPC-0767 is administered with venetoclax.

In embodiments, MPC-0767 is administered following an initial therapy comprising a DNA methyltransferase inhibitor such as 5′azacytidine or decitabine. In embodiments, the MPC-0767 is administered either alone or in combination with the DNA methyltransferase inhibitor.

In embodiments, the disclosure also provides methods of combination therapy comprising MPC-0767 in combination with one or more additional API(s) selected from anthracyclines, such as daunorubicin, doxorubicin, epirubicin, mitoxantrone, and idarubicin; cytarabine; tyrosine kinase inhibitors (TKI) such as midostaurin, sorefenib, crenolanib, quizartinib, tandutinib, gilteritinib, lestaurtinib, dovitinib, pacritinib, and XL999; etoposide, fludarabine, G-CSF, azacytidine, decitabine, venetoclax, ABT-737, navitoclax, obatoclax, sabutoclax, S 55746, AT-101 (Gossypol), and APG-1252, and combinations of any of the foregoing.

In embodiments, the one or more additional API(s) for administration in combination therapy with MPC-0767 is selected from arsenic trioxide (trisenox), cerubidine (Daunorubicin Hydrochloride), clafen (Cyclophosphamide), cyclophosphamide, cytarabine (tarabine PFS), cytosar-U (Cytarabine), cytoxan (Cyclophosphamide), daunorubicin hydrochloride (rubidomycin), doxorubicin hydrochloride, enasidenib mesylate, idamycin (idarubicin hydrochloride), idarubicin hydrochloride idhifa (Enasidenib Mesylate), midostaurin (Rydapt), mitoxantrone hydrochloride, neosar (Cyclophosphamide), thioguanine (Tabloid), vincristine sulfate (vincasar PFS), azacytidine, and decitabine, and combinations of any of the foregoing.

In embodiments, the additional API(s) is a PD-1/PD-L1 inhibitor or a Bcl-2 pathway inhibitor. In embodiments, the PD-1/PD-L1 inhibitor is selected from the group consisting of AMP-224, AMP-514/MEDI-0680, atezolizumab (MPDL3280A), avelumab (MSB0010718C), BGB-A317, BMS936559, cemiplimab (REGN2810), durvalumab (MEDI-4736), JTX-4014, nivolumab (BMS-936558), pembrolizumab (Keytruda, MK-3475), and SHR-1210.

In embodiments, the Bcl-2 pathway inhibitor is selected from the group consisting of ABT-737, AT-101 (Gossypol), APG-1252, A1155463, A1210477, navitoclax, obatoclax, sabutoclax, venetoclax, S 55746, and WEHI-539. In embodiments, the Bcl-2 pathway inhibitor is an inhibitor of BCL2, BCLXL, or MCL1. In embodiments, the Bcl-2 pathway inhibitor is selected from AMG-176, MIK665 and S641315. In embodiments, the Bcl-2 pathway inhibitor is selected from ABT-737, navitoclax, and venetoclax. In embodiments, the Bcl-2 pathway inhibitor is venetoclax.

In embodiments, the Raf inhibitor is selected from PLX4032 (vemurafenib), sorafenib, PLX-4720, GSK2118436 (dabrafenib), GDC-0879, RAF265, AZ 628, NVP-BHG712, SB90885, ZM 336372, GW5074, TAK-632, CEP-32496 and LGX818 (Encorafenib). In embodiments, the Raf inhibitor is a polypeptide (e.g., an antibody or fragment thereof) or nucleic acid (e.g., a double-stranded small interfering RNA, a short hairpin RNA, a micro-RNA, an antisense oligonucleotide, a morpholino, a locked nucleic acid, or an aptamer) that binds to and inhibits the expression level or activity of a Raf (e.g., A-Raf, B-Raf, C-Raf) or a nucleic acid encoding the Raf protein.

In embodiments, the EZH2 inhibitor is selected from GSK343, EPZ6438 (Tazemetostat), CPI-1205, GSK2816126, and PF-06821497.

In embodiments, the AML is characterized by an FLT3-ITD mutation and the method comprises venetoclax as the additional API.

In embodiments, the subject in need of treatment is one whose cancer is refractory to, or has relapsed after, treatment with gilteritinib, midostaurin, or sorafenib.

Chronic lymphocytic leukemia (CLL)

CLL is one of the most common types of leukemia in adults. It is characterized by progressive accumulation of abnormal lymphocytes. About 10% of untreated CLL patients carry a 17p chromosomal deletion which removes tumor suppressor activity. This mutation occurs in about 20% of patients having relapsed CLL. Oral venetoclax has been approved by the US Food and Drug Administration for the treatment of CLL in patients who have relapsed or refractory cancer and carry the 17p mutation.

As discussed above and shown in more detail infra, MPC-0767 in combination with venetoclax showed remarkable synergistic activity. These results suggest that MPC-0767 may be particularly effective when administered in combination with a Bcl-2 inhibitor. As noted above and described further in the examples, MPC-0767 also abrogates interferon gamma induced PD-1 expression in primary AML cells, suggesting that MPC-0767 may also be particularly effective in combination with PD-1/PD-L1 inhibitors. Accordingly, the disclosure also provides methods of treating CLL in a subject in need thereof by administering to the subject a therapeutically effective amount of MPC-0767 in combination with one or more additional API(s). In embodiments, the additional API(s) is a PD-1/PD-L1 inhibitor or a Bcl-2 pathway inhibitor. In embodiments, the PD-1/PD-L1 inhibitor selected from the group consisting of AMP-224, AMP-514/MEDI-0680, atezolizumab (MPDL3280A), avelumab (MSB0010718C), BGB-A317, BMS936559, cemiplimab (REGN2810), durvalumab (MEDI-4736), JTX-4014, nivolumab (BMS-936558), pembrolizumab (Keytruda, MK-3475), and SHR-1210. In embodiments, the Bcl-2 pathway inhibitor is selected from the group consisting of ABT-737, AT-101 (Gossypol), APG-1252, A1155463, A1210477, navitoclax, obatoclax, sabutoclax, venetoclax, S 55746, and WEHI-539. In embodiments, the Bcl-2 pathway inhibitor is an inhibitor of BCL2, BCLXL, or MCL1. In embodiments, the Bcl-2 pathway inhibitor is selected from AMG-176, MIK665 and 5641315. In embodiments, the Bcl-2 pathway inhibitor is selected from ABT-737, navitoclax, and venetoclax. In embodiments, the Bcl-2 pathway inhibitor is venetoclax.

### Non-Small Cell Lung Cancer (NSCLC)

EGFR and HER2 are transmembrane protein kinase receptors which initiate intracellular signal transduction pathways regulating cell differentiation, proliferation, motility, and survival. Aberrant activation of these receptors can arise through point mutations, deletions or insertions resulting in constitutive signaling by the receptor and activation of the attendant pathways. Aberrant activation of these receptors is directly linked to oncogenesis in various types of cancer, including NSCLC.

Both EGFR and HER2 are also client proteins of HSP90. EGFR and HER2 have each been shown to be degraded in a proteasome-dependent manner upon treatment with HSP90 inhibitors.

About 4-20% of NSCLC are characterized by EGFR ins20 mutations. Cancers having these mutations are generally also refractory to EGFR-targeted therapies, or relapse following such therapies, including EGFR-targeted PKIs.

Accordingly, the present disclosure provides methods which seek to exploit the dependence of certain NSCLC cancers on HSP90 to stabilize mutant EGFR and HER, through the use of pharmacological inhibition of HSP90. In particular, the methods exploit the susceptibility of NSCLC tumors harboring mutations in exon20 of EGFR and/or HER2.

In embodiments, the disclosure provides methods of treating NSCLC in a subject in need of such treatment, the methods comprising administering MPC-0767, or a pharmaceutically acceptable salt thereof, to the subject. In embodiments, the subject is one having a cancer that is non-responsive or refractory to, or has relapsed after, treatment with a ‘standard of care’ or first-line therapeutic agent against NSCLC.

In embodiments, the disclosure also provides methods of treating NSCLC based on combination therapy with MPC-0767 and one or more additional APIs, as discussed above. In embodiments the additional API(s) is selected from afatinib, AP32788, poziotinib, osimertinib, erlotinib, gefitinib, bragatinib, dacomitinib, lapatinib, AP32788, crizotinib, brigatinib, ceritinib, alectinib, AP26113, PF-06463922, X-396, RXDX-101, dabrafenib, tremetinib, nintedanib, abemaciclib, ABP 215, bevacizumab, ramucirumab, necitumumab, ipilimumab, denosumab, tremelimumab, bavituximab, nivulomab, atezolizumab, pembrolizumab, avelumab, durvalumab, carboplatin, cisplatin, docetaxel, gemcitabine, Nab-paclitaxel, paclitaxel (Taxol), pemetrexed, vinorelbine, etoposide, aldoxorubicin, topotecan, irinotecan, and combinations of any of the foregoing.

### Therapeutically Effective Amounts of MPC-0767

In the context of the methods described herein, the amount of MPC-0767 administered to the subject is a therapeutically effective amount. The term “therapeutically effective amount” refers to an amount sufficient to treat, ameliorate a symptom of, reduce the severity of, or reduce the duration of the disease or disorder being treated or, in the context of combination therapies, it may also include the amount capable of improving the therapeutic effect of another therapy or active pharmaceutical ingredient. In the context of the present disclosure, the therapeutically effective amount is the amount sufficient to treat a cancer in a subject in need of such treatment, as described here.

In embodiments, the therapeutically effective amount of MPC-0767, or a pharmaceutically acceptable salt thereof, is in the range of 0.01 mg/kg to 100 mg/kg per day based on the total body weight of a human subject, in single or divided doses. In embodiments, the range is from 10-1000 mg or from 50-500 mg delivered one, twice, or three times daily.

In embodiments, the therapeutically effective amount is about 10 mg, about 50 mg, about 75 mg, about 100 mg, about 250 mg, about 500 mg, about 750 mg, or about 1000 mg delivered one, twice, or three times daily.

In embodiments, the therapeutically effective amount is about 50 mg, about 75 mg, about 100 mg, about 200 mg, about 300 mg, about 400 mg, or about 500 mg, delivered once, twice, or three times daily.

In embodiments, the therapeutically effective amount of MPC-0767, or a pharmaceutically acceptable salt thereof, preferably a mesylate salt, is the amount sufficient to achieve a plasma Cₘₐₓ in the subject with daily dosing ranging from 1,500 ng/ml to 30,000 ng/ml, preferably from 6,000 ng/ml to 30,000 ng/ml or from 6,000 ng/ml to 15,000 ng/ml.

### Treating Cancer

As used herein, “treatment”, “treating”, or “treat” describes the management and care of a patient for the purpose of combating a disease, condition, or disorder and includes the administration of MPC-0767 to alleviate the symptoms or complications of a disease, condition or disorder, or to eliminate the disease, condition or disorder.

In embodiments of any of the methods described here, including both monotherapy with MPC-0767 and combination therapies with one or more additional APIs, the administration of MPC-0767 or combinations thereof leads to the elimination of a symptom or complication of the cancer being treated, however elimination of the cancer is not required. In one embodiment, the severity of the symptom is decreased. In the context of cancer, such symptoms may include clinical markers of severity or progression including the degree to which a tumor secretes growth factors, degrades the extracellular matrix, becomes vascularized, loses adhesion to juxtaposed tissues, or metastasizes, as well as the number of metastases and reduction in tumor size and/or volume.

Treating cancer according to the methods described herein can result in a reduction in size of a tumor. A reduction in size of a tumor may also be referred to as “tumor regression.” Preferably, after treatment, tumor size is reduced by 5% or greater relative to its size prior to treatment; more preferably, tumor size is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Size of a tumor may be measured by any reproducible means of measurement. The size of a tumor may be measured as a diameter of the tumor.

Treating cancer according to the methods described herein can result in a reduction in tumor volume. Preferably, after treatment, tumor volume is reduced by 5% or greater relative to its size prior to treatment; more preferably, tumor volume is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Tumor volume may be measured by any reproducible means of measurement.

Treating cancer according to the methods described herein can result in a decrease in number of tumors. Preferably, after treatment, tumor number is reduced by 5% or greater relative to number prior to treatment; more preferably, tumor number is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. Number of tumors may be measured by any reproducible means of measurement. The number of tumors may be measured by counting tumors visible to the naked eye or at a specified magnification. Preferably, the specified magnification is 2×, 3×, 4×, 5×, 10×, or 50×. For hematologic cancers, the count may be the number of cells related to the cancer (e.g., lymphoma or leukemia cells) in a sample of blood.

Treating cancer according to the methods described herein can result in a decrease in the number of metastatic lesions in other tissues or organs distant from the primary tumor site. Preferably, after treatment, the number of metastatic lesions is reduced by 5% or greater relative to the number prior to treatment; more preferably, the number of metastatic lesions is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. The number of metastatic lesions may be measured by any reproducible means of measurement. The number of metastatic lesions may be measured by counting metastatic lesions visible to the naked eye or at a specified magnification. Preferably, the specified magnification is 2×, 3×, 4×, 5×, 10×, or 50×.

Treating cancer according to the methods described herein can result in an increase in average survival time of a population of treated subjects in comparison to a population receiving carrier alone. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment. An increase in average survival time of a population may also be measured, for example, by calculating for a population the average length of survival following completion of a first round of treatment.

Treating cancer according to the methods described herein can result in an increase in average survival time of a population of treated subjects in comparison to a population of untreated subjects. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment. An increase in average survival time of a population may also be measured, for example, by calculating for a population the average length of survival following completion of a first round of treatment.

Treating cancer according to the methods described herein can result in an increase in average survival time of a population of treated subjects in comparison to a population receiving monotherapy with a drug that is not MPC-0767. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment. An increase in average survival time of a population may also be measured, for example, by calculating for a population the average length of survival following completion of a first round of treatment.

Treating cancer according to the methods described herein can result in a decrease in the mortality rate of a population of treated subjects in comparison to a population receiving carrier alone. Treating a disorder, disease or condition according to the methods described herein can result in a decrease in the mortality rate of a population of treated subjects in comparison to an untreated population. Treating a disorder, disease or condition according to the methods described herein can result in a decrease in the mortality rate of a population of treated subjects in comparison to a population receiving monotherapy with a drug that is not MPC-0767. Preferably, the mortality rate is decreased by more than 2%; more preferably, by more than 5%; more preferably, by more than 10%; and most preferably, by more than 25%. A decrease in the mortality rate of a population of treated subjects may be measured by any reproducible means. A decrease in the mortality rate of a population may be measured, for example, by calculating for a population the average number of disease-related deaths per unit time following initiation of treatment. A decrease in the mortality rate of a population may also be measured, for example, by calculating for a population the average number of disease-related deaths per unit time following completion of a first round of treatment.

Treating cancer according to the methods described herein can result in a decrease in tumor growth rate. Preferably, after treatment, tumor growth rate is reduced by at least 5% relative to number prior to treatment; more preferably, tumor growth rate is reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Tumor growth rate may be measured by any reproducible means of measurement. Tumor growth rate can be measured according to a change in tumor diameter per unit time. In one embodiment, after treatment the tumor growth rate may be about zero and is determined to maintain the same size, e.g., the tumor has stopped growing.

Treating cancer according to the methods described herein can result in a decrease in tumor regrowth. Preferably, after treatment, tumor regrowth is less than 5%; more preferably, tumor regrowth is less than 10%; more preferably, less than 20%; more preferably, less than 30%; more preferably, less than 40%; more preferably, less than 50%; even more preferably, less than 50%; and most preferably, less than 75%. Tumor regrowth may be measured by any reproducible means of measurement. Tumor regrowth is measured, for example, by measuring an increase in the diameter of a tumor after a prior tumor shrinkage that followed treatment. A decrease in tumor regrowth is indicated by failure of tumors to reoccur after treatment has stopped.

### Pharmaceutical Compositions and Formulations

The present disclosure provides pharmaceutical compositions comprising an amount of MPC-0767, or a pharmaceutically acceptable salt thereof, preferably a mesylate salt, either alone or in combination with an additional API. In accordance with any of the embodiments described here, the pharmaceutical composition may be adapted for oral, buccal, or parenteral administration. In embodiments, the pharmaceutical composition may be adapted for pulmonary administration, for example by inhalation. In embodiments, the pharmaceutical composition is adapted for oral administration. In embodiments, the pharmaceutical composition is adapted for parenteral administration.

In embodiments, the MPC-0767 or a pharmaceutically acceptable salt thereof, preferably a mesylate salt, is combined with at least one additional API in a single dosage form. In embodiments, the at least one additional API is selected from an agent described supra in connection with methods of treatment using combination therapy.

A “pharmaceutical composition” is a formulation containing the compounds described herein in a pharmaceutically acceptable form suitable for administration to a subject. As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. Examples of pharmaceutically acceptable excipients include, without limitation, sterile liquids, water, buffered saline, ethanol, polyol (for example, glycerol, propylene glycol, liquid polyethylene glycol and the like), oils, detergents, suspending agents, carbohydrates (e.g., glucose, lactose, sucrose or dextran), antioxidants (e.g., ascorbic acid or glutathione), chelating agents, low molecular weight proteins, or suitable mixtures thereof.

A pharmaceutical composition can be provided in bulk or in dosage unit form. It is especially advantageous to formulate pharmaceutical compositions in dosage unit form for ease of administration and uniformity of dosage. The term “dosage unit form” as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved. A dosage unit form can be an ampoule, a vial, a suppository, a dragee, a tablet, a capsule, an IV bag, or a single pump on an aerosol inhaler.

In therapeutic applications, the dosages vary depending on the agent, the age, weight, and clinical condition of the recipient patient, and the experience and judgment of the clinician or practitioner administering the therapy, among other factors affecting the selected dosage. Generally, the dose should be a therapeutically effective amount. Dosages can be provided in mg/kg/day units of measurement (which dose may be adjusted for the patient's weight in kg, body surface area in m², and age in years). An effective amount of a pharmaceutical composition is that which provides an objectively identifiable improvement as noted by the clinician or other qualified observer. For example, alleviating a symptom of a disorder, disease or condition. As used herein, the term “dosage effective manner” refers to an amount of a pharmaceutical composition to produce the desired biological effect in a subject or cell.

For example, the dosage unit form can comprise 1 nanogram to 2 milligrams, or 0.1 milligrams to 2 grams; or from 10 milligrams to 1 gram, or from 50 milligrams to 500 milligrams or from 1 microgram to 20 milligrams; or from 1 microgram to 10 milligrams; or from 0.1 milligrams to 2 milligrams.

The pharmaceutical compositions can take any suitable form (e.g, liquids, aerosols, solutions, inhalants, mists, sprays; or solids, powders, ointments, pastes, creams, lotions, gels, patches and the like) for administration by any desired route (e.g, pulmonary, inhalation, intranasal, oral, buccal, sublingual, parenteral, subcutaneous, intravenous, intramuscular, intraperitoneal, intrapleural, intrathecal, transdermal, transmucosal, rectal, and the like). For example, a pharmaceutical composition of the disclosure may be in the form of an aqueous solution or powder for aerosol administration by inhalation or insufflation (either through the mouth or the nose), in the form of a tablet or capsule for oral administration;; in the form of a sterile aqueous solution or dispersion suitable for administration by either direct injection or by addition to sterile infusion fluids for intravenous infusion; or in the form of a lotion, cream, foam, patch, suspension, solution, or suppository for transdermal or transmucosal administration.

A pharmaceutical composition can be in the form of an orally acceptable dosage form including, but not limited to, capsules, tablets, buccal forms, troches, lozenges, and oral liquids in the form of emulsions, aqueous suspensions, dispersions or solutions. Capsules may contain mixtures of a compound of the present disclosure with inert fillers and/or diluents such as the pharmaceutically acceptable starches (e.g., corn, potato or tapioca starch), sugars, artificial sweetening agents, powdered celluloses, such as crystalline and microcrystalline celluloses, flours, gelatins, gums, etc. In the case of tablets for oral use, carriers which are commonly used include lactose and corn starch. Lubricating agents, such as magnesium stearate, can also be added. For oral administration in a capsule form, useful diluents include lactose and dried corn starch. When aqueous suspensions and/or emulsions are administered orally, the compound of the present disclosure may be suspended or dissolved in an oily phase is combined with emulsifying and/or suspending agents. If desired, certain sweetening and/or flavoring and/or coloring agents may be added.

A pharmaceutical composition can be in the form of a tablet. The tablet can comprise a unit dosage of a compound of the present disclosure together with an inert diluent or carrier such as a sugar or sugar alcohol, for example lactose, sucrose, sorbitol or mannitol. The tablet can further comprise a non-sugar derived diluent such as sodium carbonate, calcium phosphate, calcium carbonate, or a cellulose or derivative thereof such as methyl cellulose, ethyl cellulose, hydroxypropyl methyl cellulose, and starches such as corn starch. The tablet can further comprise binding and granulating agents such as polyvinylpyrrolidone, disintegrants (e.g. swellable crosslinked polymers such as crosslinked carboxymethylcellulose), lubricating agents (e.g. stearates), preservatives (e.g. parabens), antioxidants (e.g. BHT), buffering agents (for example phosphate or citrate buffers), and effervescent agents such as citrate/bicarbonate mixtures.

The tablet can be a coated tablet. The coating can be a protective film coating (e.g. a wax or varnish) or a coating designed to control the release of the active agent, for example a delayed release (release of the active after a predetermined lag time following ingestion) or release at a particular location in the gastrointestinal tract. The latter can be achieved, for example, using enteric film coatings such as those sold under the brand name Eudragit®.

Tablet formulations may be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, talc, sodium lauryl sulfate, microcrystalline cellulose, carboxymethylcellulose calcium, polyvinylpyrrolidone, gelatin, alginic acid, acacia gum, xanthan gum, sodium citrate, complex silicates, calcium carbonate, glycine, dextrin, sucrose, sorbitol, dicalcium phosphate, calcium sulfate, lactose, kaolin, mannitol, sodium chloride, talc, dry starches and powdered sugar. Preferred surface modifying agents include nonionic and anionic surface modifying agents. Representative examples of surface modifying agents include, but are not limited to, poloxamer 188, benzalkonium chloride, calcium stearate, cetostearyl alcohol, cetomacrogol emulsifying wax, sorbitan esters, colloidal silicon dioxide, phosphates, sodium dodecylsulfate, magnesium aluminum silicate and triethanolamine.

A pharmaceutical composition can be in the form of a hard or soft gelatin capsule. In accordance with this formulation, the compound of the present disclosure may be in a solid, semi-solid, or liquid form.

A pharmaceutical composition can be in the form of a sterile aqueous solution or dispersion suitable for parenteral administration. The term parenteral as used herein includes subcutaneous, intracutaneous, intravenous, intramuscular, intra-articular, intraarterial, intrasynovial, intrasternal, intrathecal, intralesional and intracranial injection or infusion techniques.

A pharmaceutical composition can be in the form of a sterile aqueous solution or dispersion suitable for administration by either direct injection or by addition to sterile infusion fluids for intravenous infusion, and comprises a solvent or dispersion medium containing, water, ethanol, a polyol (e.g., glycerol, propylene glycol and liquid polyethylene glycol), suitable mixtures thereof, or one or more vegetable oils. Solutions or suspensions of the compound of the present disclosure as a free base or pharmacologically acceptable salt can be prepared in water suitably mixed with a surfactant. Examples of suitable surfactants are given below. Dispersions can also be prepared, for example, in glycerol, liquid polyethylene glycols and mixtures of the same in oils.

The pharmaceutical compositions for use in the methods of the present disclosure can further comprise one or more additives in addition to any carrier or diluent (such as lactose or mannitol) that is present in the formulation. The one or more additives can comprise or consist of one or more surfactants. Surfactants typically have one or more long aliphatic chains such as fatty acids which enables them to insert directly into the lipid structures of cells to enhance drug penetration and absorption. An empirical parameter commonly used to characterize the relative hydrophilicity and hydrophobicity of surfactants is the hydrophilic-lipophilic balance (“HLB” value). Surfactants with lower HLB values are more hydrophobic, and have greater solubility in oils, while surfactants with higher HLB values are more hydrophilic, and have greater solubility in aqueous solutions. Thus, hydrophilic surfactants are generally considered to be those compounds having an HLB value greater than about 10, and hydrophobic surfactants are generally those having an HLB value less than about 10. However, these HLB values are merely a guide since for many surfactants, the HLB values can differ by as much as about 8 HLB units, depending upon the empirical method chosen to determine the HLB value.

Among the surfactants for use in the compositions of the disclosure are polyethylene glycol (PEG)-fatty acids and PEG-fatty acid mono and diesters, PEG glycerol esters, alcohol-oil transesterification products, polyglyceryl fatty acids, propylene glycol fatty acid esters, sterol and sterol derivatives, polyethylene glycol sorbitan fatty acid esters, polyethylene glycol alkyl ethers, sugar and its derivatives, polyethylene glycol alkyl phenols, polyoxyethylene-polyoxypropylene (POE-POP) block copolymers, sorbitan fatty acid esters, ionic surfactants, fat-soluble vitamins and their salts, water-soluble vitamins and their amphiphilic derivatives, amino acids and their salts, and organic acids and their esters and anhydrides.

The present disclosure also provides packaging and kits comprising pharmaceutical compositions for use in the methods of the present disclosure. The kit can comprise one or more containers selected from the group consisting of a bottle, a vial, an ampoule, a blister pack, and a syringe. The kit can further include one or more of instructions for use in treating and/or preventing a disease, condition or disorder of the present disclosure, one or more syringes, one or more applicators, or a sterile solution suitable for reconstituting a pharmaceutical composition of the present disclosure.

All percentages and ratios used herein, unless otherwise indicated, are by weight. Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

### EXAMPLES

As shown in the examples described below, treatment of AML cells or lung cancer cells with MPC-0767 leads to decreased cell viability and destabilization of the key oncogenic receptor. MPC-0767 demonstrates preferential cytotoxicity toward AML cell lines and primary cells expressing activating mutations in FLT3, compared to cells not having the activating mutations, both in vitro and in a mouse xenograft model. In addition, the experiments below show that while AML cells cultured with conditioned media from stromal cells become resistant to various FLT3 inhibitors, they remain sensitive to MPC-0767. Since the development of drug resistance is a critical limitation of protein kinase inhibitor therapy generally, and FLT3 inhibitor therapy in particular, the sensitivity of resistant AML cells to MPC-0767 indicates that MPC-0767 is an exciting new option for the treatment of AML. The data provided here show that HSP90 inhibitors such as MPC-0767 can have clinical efficacy in patients with AML that harbor activating mutations in FLT3. Moreover, in AML cells that are resistant to FLT3 inhibitors due to secondary mutations in FLT3 itself or activation of a different signaling pathway(s), MPC-0767 retains cytotoxic activity. This indicates that HSP90 inhibitors such as MPC-0767 can have clinical efficacy in patients with AML that are relapsed after treatment with FLT3 inhibitors, or refractory to FLT3 inhibitors. MPC-0767 also shows synergy with therapies that are either already established, or are still being investigated for the treatment of AML. MPC-0767 also showed a surprising highly synergistic activity with venetoclax across multiple cell lines in vitro and potent combinatorial activity in a systemic survival xenograft study using FLT3-ITD AML cells. Taken together, these results support MPC-0767 as an attractive new therapy for treating AML and other cancers, both as monotherapy and in combination with other APIs.

### Example 1: MPC-0767 Inhibits Cell Viability in NSCLC Cell Lines Carrying Mutations in EGFR and HER2

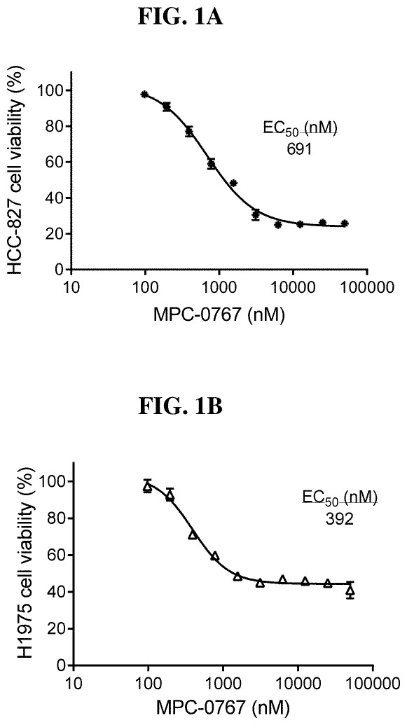

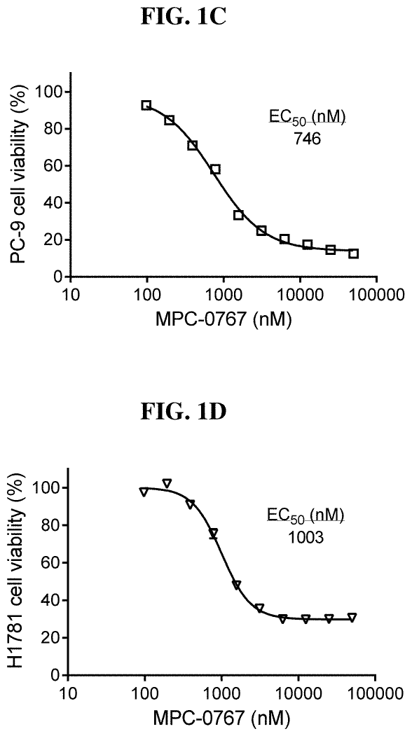

The NSCLC cell lines HCC-827 (EGFR L858R), H1975 (EGFR L858R/T790M) PC-9 (EGFR Del E746_A750) and H1781 (HER2 G7776insV G/C) were treated with MPC-0767 at a concentration range of 98-50000 nM for 3 days, after which time cell viability was determined using CellTiter-Glo® reagent. FIG. 1 shows the dose-response curves of HCC-827 (FIG. 1A), H1975 (FIG. 1B), PC-9 (FIG. 1C) and H1781 (FIG. 1D) cell lines. All EC₅₀ values were within clinically achievable concentrations.

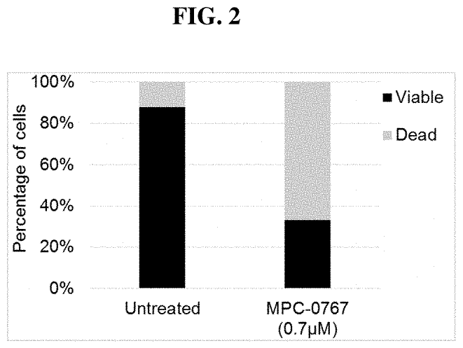

To verify the mechanism of the loss of cell viability, H1975 cells were treated with MPC-0767 (0.7 μM) for 72 hours. After this time, cells were stained with 7-amino-actinomycin D (7-AAD) and annexin V, markers of cell membrane integrity and of apoptosis, respectively. As shown in FIG. 2, treatment of H1975 cells with MPC-0767 (0.7 μM) resulted in a decrease in the percentage of viable cells (7-AAD negative and annexin V negative) and an increase in the percentage of cells displaying markers of cell death, specifically dead (7-AAD only positive), early apoptotic (annexin V only positive), or late stage apoptotic/necrotic (7-ADD and annexin V positive).

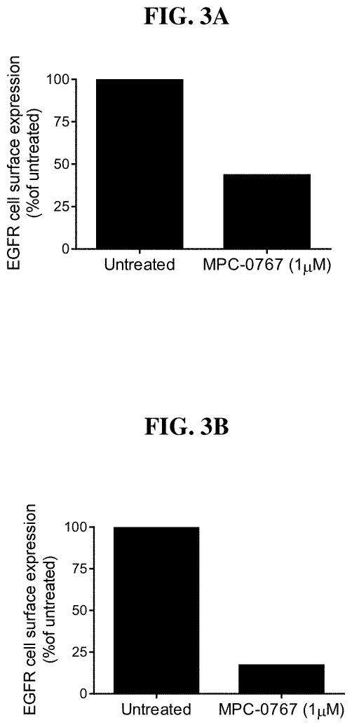

FIG. 3 shows that MPC-0767 (1 μM) decreased mutant EGFR on the cell surface of H1975 (A) and PC-9 (B) cells when treated for 24 hours. These findings confirm that MPC-0767 targets and degrades EGFR in lung cancer cell lines.

To determine whether MPC-0767 can also promote degradation of an EGFR exon20ins mutant, the BaF3 murine cell line was used (Warmuth et al., Curr Opin Oncol., 200719: 55-60). This cell line is dependent upon exogenous IL-3 for survival/growth but upon introduction of an oncogene, the cells no longer depend on exogenous IL-3, and instead survival is driven by the introduced oncogene. Thus, drugs that target the introduced oncogene will reduce BaF3 cell viability providing a mechanism to screen small molecules against relevant oncogenic mutations that arise in the clinic.

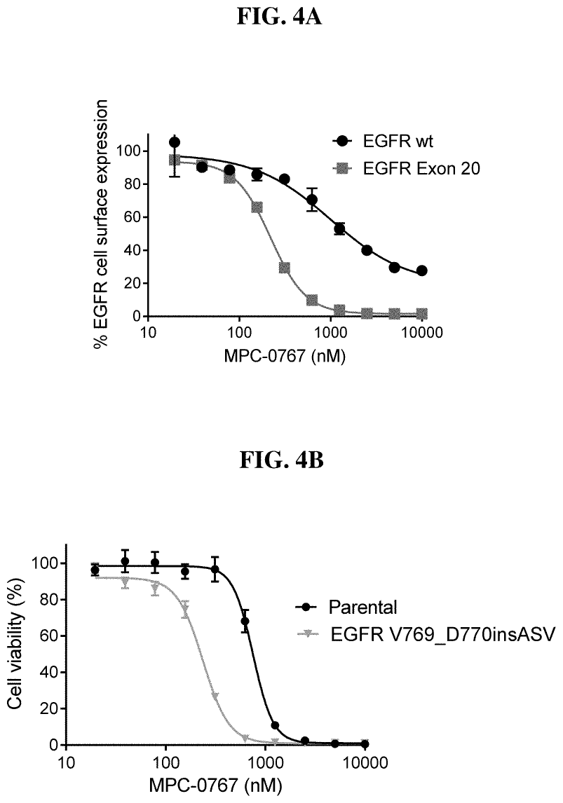

BaF3 cells harboring EGFR wild type (WT) or EGFR exon20 V769_D770insASV mutant were treated with increasing concentrations of MPC-0767 for 24 hours. After this time, cells were harvested for flow cytometry to assess cell surface EGFR expression (antibody for detection recognized both WT and mutant proteins). As shown in FIG. 4A, MPC-0767 was able to reduce EGFR WT (EC₅₀=1 μM), but was more potent toward the EGFR exon20 V769_D770insASV mutant (EC₅₀=0.2 μM). We further tested whether this finding translated to reduced survival in BaF3 cells expressing the EGFR mutant. Parental BaF3 cells (no mutant) or cells harboring EGFR exon20 V769_D770insASV were treated with increasing concentrations of MPC-0767 for 72 hours after which cell viability was determined using CellTiter-Glo®. FIG. 4B shows that BaF3 cells harboring the EGFR exon20 V769_D770insASV mutant are more reliant on HSP90 since they are approximately 3 times more sensitive to MPC-0767 than parental cells (parental EC₅₀=753 nM, EGFR exon20 V769_D770insASVmutant EC₅₀=236 nM).

Collectively, the data suggest that MPC-0767 is efficacious against NSCLC driven by aberrant activation of EGFR or HER2, through degradation of the key oncogenic drivers. Moreover, given the increased reliance of mutant proteins on HSP90, MPC-0767 is more active on mutant EGFR resulting in enhanced degradative and anti-tumor activity.

### Example 2: MPC-0767 Displays Potent Anti-Leukemic Activity in AML Cells Harboring FLT3-ITD

Exponentially growing cell lines were counted and seeded into 96-well clear, flat-bottomed polystyrene microtiter plates in a final volume of 90 μL per well. For primary AML samples, cells were seeded into 384 well plates at a density of 2×10⁴ cells in a final volume of 27 μL per well. To treat cell lines or primary samples 10 μL or 3 μL, respectively, of 10× concentrations of MPC-0767, were then added to the cells to give a final concentration of 10000 nM, 5000 nM, 2500 nM, 1250 nM, 625 nM, 313 nM, 156 nM, 78 nM, 39 and 20 nM. For comparison, cells were treated with the FLT3 inhibitor gilteritinib (of 100 nM, 50 nM, 25 nM, 12.5 nM, 6.3 nM, 3.1 nM, 1.6 nM, 0.8 nM, 0.4 and 0.2 nM). Cells were seeded and treated in duplicate. After incubation for three days, cell viability was determined by measuring intracellular ATP levels using the CellTiter-Glo® assay system by adding to each well either 100 μL for 96 well plates, or 30 μL for 384 well plates. Luminescence was detected using a plate reader.

The effect of drugs on cell viability was calculated by comparing the ATP levels (luminescence counts per second) of cells exposed to test compound with those of cells exposed to vehicle (DMSO) alone. The half-maximal effective concentration (EC₅₀) for each cell line was determined using the R DRC package (R Core Team, 2017). In brief, the dose-response curves were fitted with a four-parameter logistic regression model (LL.4) according to (Eq-1) and the absolute EC₅₀ was estimated using a confidence interval of 0.95.

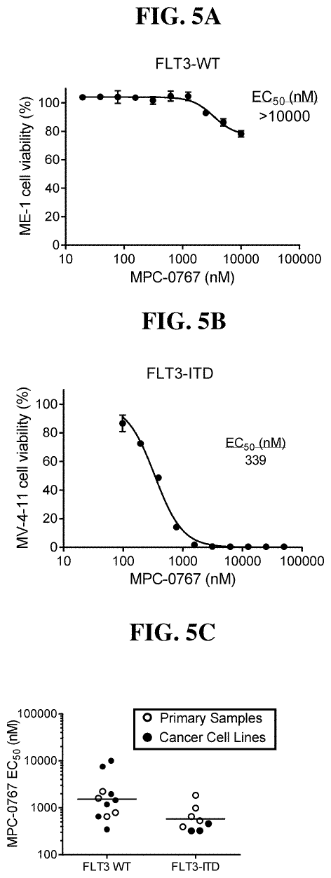

FIG. 5A shows a representative dose-response curve from a cell line (ME1), which expresses the wild type (WT) FMS-like tyrosine kinase 3 (FLT3) protein, while FIG. 5B shows a representative dose-response curve from a cell line (MV-4-11) which harbors FLT3 internal tandem duplication (FLT3 ITD). To further illustrate that MPC-0767 has greater efficacy in AML cells harboring FLT3-ITD than in FLT3 WT, the anti-leukemic activity of MPC-0767 (EC₅₀ values) derived from cell lines (n=10) and primary samples (n=9) was assayed. FIG. 5C shows the output of this analysis where the geometric mean EC₅₀ value was 1525 nM for FLT3 WT cells (n=11) as compared with 576 nM for FLT3-ITD cells (n=8). These data suggest that MPC-0767 displays enhanced activity toward AML cells harboring FLT3-ITD and a subset of AML cells with WT FLT3.

### Example 3: MPC-0767 is Cytotoxic in Primary AML Cells Harboring FLT3-ITD

To test whether the anti-leukemic effect of MPC-0767 is due to induction of cell death, 4 primary AML samples (all harboring FLT3-ITD) were treated with increasing concentrations of MPC-0767 for 72 hours. Samples were then processed for quantification by flow cytometry of cells positive for annexin V and 7AAD. These markers allow the detection of cell death, specifically dead (7AAD only positive), early apoptotic (annexin V only positive) or late stage apoptotic/necrotic (7AAD and annexin V positive) populations were combined to give a readout of cell death.

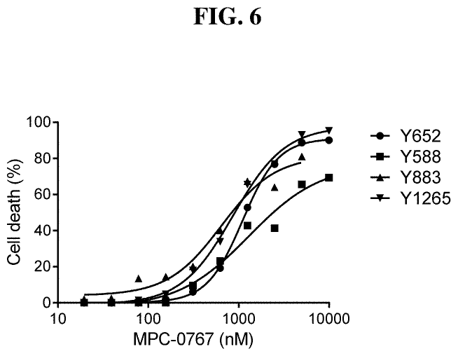

As shown in FIG. 6, primary AML samples treated with MPC-0767 show a dose-dependent increase in cell death. Of note, one of the samples (Y1265) was obtained from a patient who relapsed on gilteritinib.

These findings demonstrate that MPC-0767 induces cell death, through the induction of apoptosis, in primary AML samples that harbor FLT3-ITD. Moreover, MPC-0767 is active in cases in which the patient's tumor has relapsed gilteritinib treatment.

### Example 4: MPC-0767 Demonstrates Efficacy in Vivo

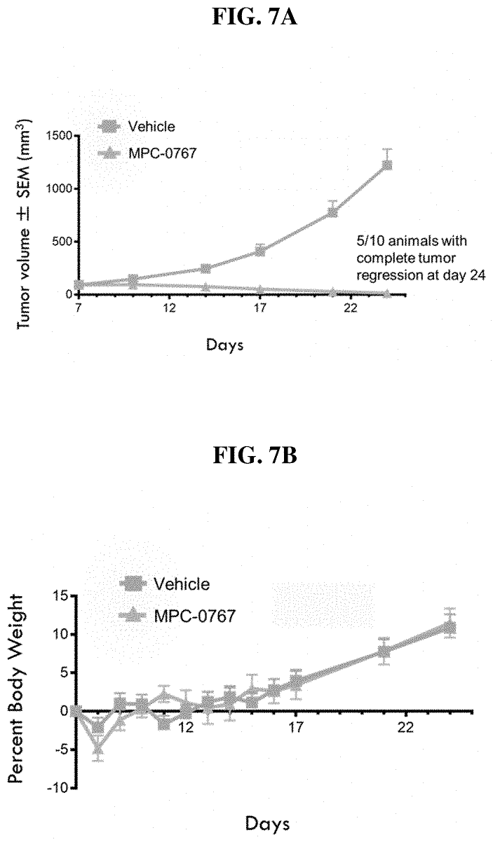

To demonstrate MPC-0767 efficacy in vivo, a xenograft study was performed using the MV-4-11 cell line. Each mouse was inoculated subcutaneously in the right flank with 5×10⁶ tumor cells in 0.1 ml PBS/Matrigel (1:1). When the mean tumor volume reached 91 mm³ in size, mice were randomized into 2 groups of 10. Mice were then dosed orally with either vehicle or with MPC-0767 200 mg/kg QD×2 days then reduced to 150 mg/kg QD×15 days. Tumor measurements (caliper) were taken on the indicated days. As shown in FIG. 7, MPC-0767 induced a tumor regression of 84% (FIG. 7A), with complete tumor regression in 5/10 animals, without significant effects on body weight (FIG. 7B). Student t-test was used to evaluate the statistical significance of the difference between these groups P<0.0001.

This data confirms that MPC-0767 displays potent anti-tumor activity in vivo.

### Example 5: MPC-0767 is Efficacious in a FLT3 Inhibitor (Midostaurin) Resistant Cell Line

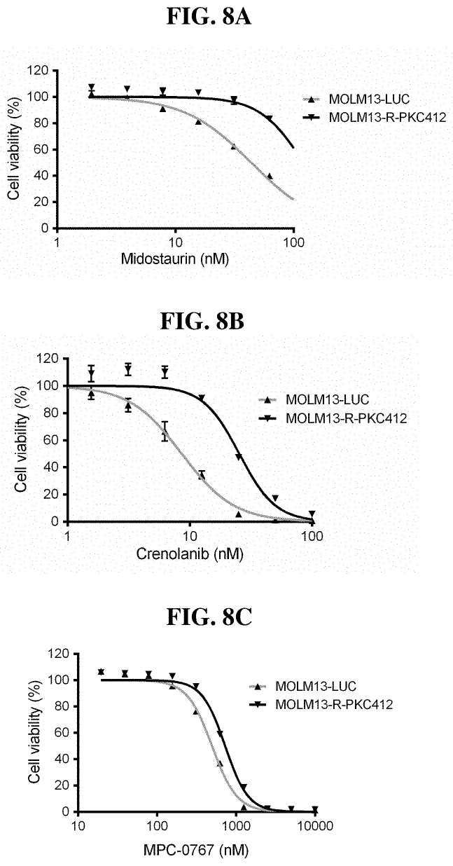

In the clinic, tyrosine kinase inhibitors that target FLT3 initially show positive responses, but patients inevitably relapse due to the development of drug-resistance through various mechanisms, as discussed above. To address whether MPC-0767 may be effective in this context of drug resistance, we utilized a cell line (MOLM-13) that had been continuously treated with midostaurin to generate a midostaurin-resistant cell line, designated MOLM-13-R-PKC412, as previously described (Weisberg et al., PLoS One, 2011). Parental MOLM-13 cells transfected with a control plasmid (MOLM-13-LUC) and MOLM-13-R-PKC412 cells were treated with midostaurin (2-100 nM), which was used to verify resistance, crenolanib (0.2-100 nM), another FLT3 inhibitor, or MPC-0767 (20-10000 nM) for 72 hours. Cell viability was assessed using CellTiter-Glo® and EC₅₀ values were determined for midostaurin, crenolanib and MPC-0767 by comparing cell viability in the presence of varying concentrations of drug to viability in the presence of vehicle (DMSO), set to 100%, using equation 1 (as described above). As shown in FIG. 8A, the midostaurin-resistant cells showed an increase in resistance to midostaurin compared to the control cell line (˜2.5 fold: MOLM-13-LUC EC₅₀=44 nM versus MOLM-13-R-PKC412 EC₅₀=112 nM). Moreover, as shown in FIG. 8B, the midostaurin-resistant cells also displayed cross resistance to another FLT3 inhibitor, crenolanib (approximately 3-fold: MOLM-13-LUC EC₅₀=9 nM versus MOLM-13-R-PKC412 EC₅₀=25 nM). In contrast, as shown in FIG. 8C, the EC₅₀ values of MPC-0767 between control cells and midostaurin-resistant cells was less than 1.5-fold (MOLM-13-LUC EC₅₀=496 nM versus MOLM-13-R-PKC412 EC₅₀=727 nM).

Taken together, these data demonstrate that MPC-0767 retains anti-leukemic activity in cells that acquire resistance to FLT3 inhibitors.

### Example 6: MPC-0767 is Efficacious Under Conditions that Confer Resistance to FLT3 Inhibitors

To determine whether MPC-0767 demonstrated efficacy against AML cells that acquired resistance via other mechanisms (non-mutational), such as stromal-induced signaling, the MOLM-14 cell line (harboring FLT3-ITD) was seeded in either regular medium (RPMI; non-stromal) or in HS-5 cell line conditioned medium. HS-5 is a human marrow stromal cell line that secretes various growth factors sufficient to support hematopoietic progenitor growth (Roecklein et al., Blood, 1995) which thus mimics stromal conditions. Cells were then treated with the FLT3 inhibitor gilteritinib (0.2-100 nM), or crenolanib (0.2-100 nM) or with MPC-0767 (20-10000 nM) for 72 hours. Cell viability was assessed using CellTiter-Glo® and EC₅₀ values were determined for gilteritinib, crenolanib and MPC-0767 in either non-stromal medium or stromal condition medium by comparing cell viability in the presence of varying concentrations of drug to viability in the presence of vehicle (DMSO), set to 100%, using equation 1 (as described above).

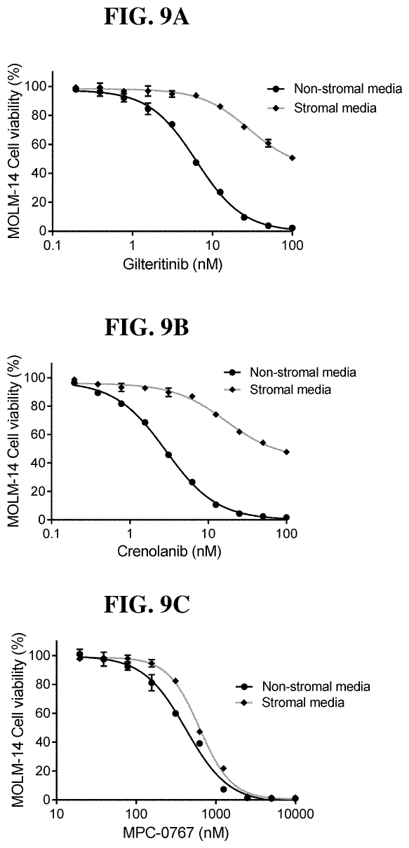

As shown in FIG. 9, MOLM-14 cells were resistant to the FLT3 inhibitors gilteritinib (FIG. 9A) and crenolanib (FIG. 9B) when grown in stromal media as compared to non-stromal medium (Gilteritinib: stromal media EC₅₀>100 nM versus non-stromal media EC₅₀=6 nM. Crenolanib: stromal media EC₅₀>100 nM versus non-stromal media EC₅₀=3 nM). In contrast, as shown in FIG. 9C, MPC-0767 retained anti-proliferative activity under both stromal and non-stromal conditions (stromal media EC₅₀=627 nM versus non-stromal media EC₅₀=423 nM).

These data demonstrate that AML FLT3-ITD cells, when grown under stromal conditions that render FLT3 inhibitors ineffective, retain sensitivity to MPC-0767.

### Example 7: MPC-0767 Degrades FLT3-ITD in AML Cell Lines

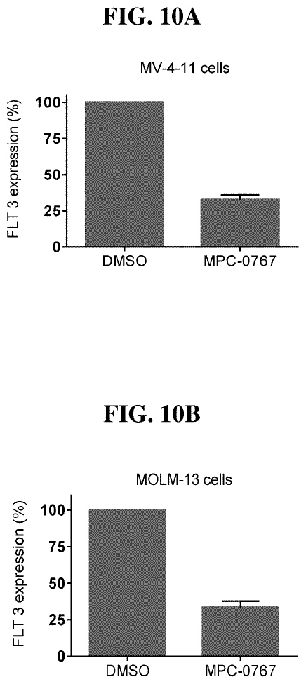

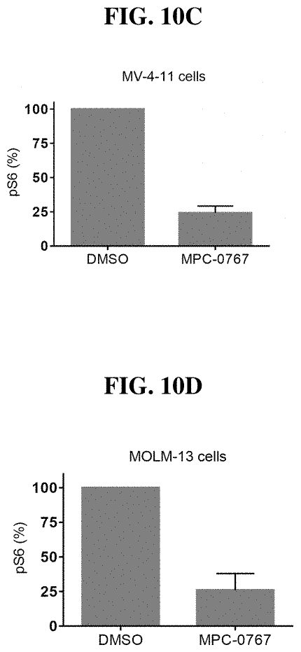

To determine whether MPC-0767 can promote the degradation of FLT3-ITD and abolish downstream signaling, MV-4-11 and MOLM-13 cells were treated with vehicle or MPC-0767 (1 μM) for 24 hours. Cells were harvested for flow cytometry to assess cell surface FLT3 protein abundance. In addition, the measurement of a key phosphorylation site of S6 (phospho-S6) was used as a marker for oncogenic FLT3-ITD signaling (Zimmerman et al., Blood. 2013 122(22): 3607-3615). Indeed, in both MV-4-11 and MOLM-13 cells treated with MPC-0767 there was a >65% reduction in cell surface FLT3 (FIGS. 10A and 10B, respectively) and >70% reduction in phospho-S6 (FIGS. 10C and 10D, respectively).

These findings confirm that MPC-0767 degrades FLT3-ITD, which subsequently attenuates oncogenic signaling as evidenced by reduced phospho-S6 signal.

### Example 8: MPC-0767 Induces Degradation of FLT3 Mutants

We next sought to determine whether MPC-0767 can also promote the degradation of other FLT3 mutants that have been reported to confer resistance to FLT3 inhibitors. To do this, we again utilized the BaF3 murine cell line into which the following FLT3 mutants were transfected: FLT3 wild-type, FLT3-ITD, D835V, FLT3-ITD D835V, D835Y, FLT3-ITD D835Y, D835H, FLT3-ITD D835H, F691L, or FLT3-ITD F691L.

After puromycin selection, cells were treated with increasing concentrations of MPC-0767 (20-10000 nM) for 24 hours and then stained for cell surface expression of FLT3 (and mutants) and the median signal expression was quantified by flow cytometry.

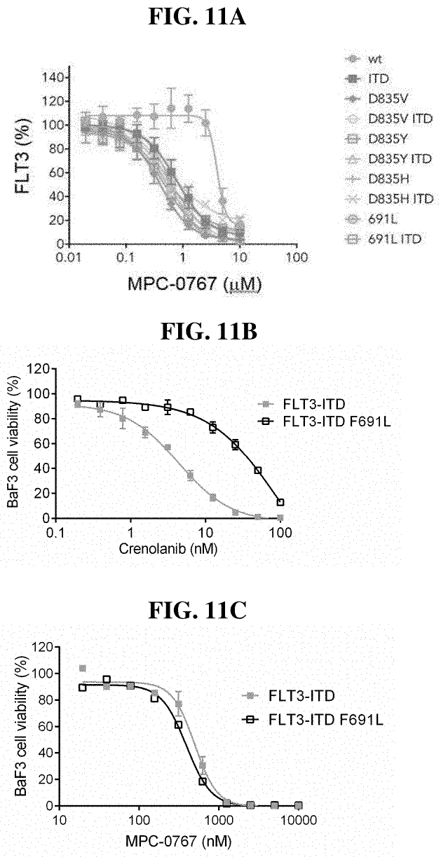

As shown in FIG. 11A, MPC-0767 reduced cell surface expression of FLT3 WT. Moreover, MPC-0767 had greater potency against FLT3 mutants (approximately 5× compared to FLT3 WT), demonstrating the greater reliance of these mutant proteins on HSP90.

The next step was to determine if MPC-0767 induced degradation of various mutant FLT3 proteins in BaF3 cells had any functional relevance. It has previously been shown that crenolanib effectively inhibits FLT3-ITD but that mutation of the gatekeeper residue F691L reduces crenolanib efficacy (Zimmerman et al., Blood, 2013 122(22): 3607-3615). Hence, MPC-0767 was tested for efficacy against the TKI-resistant FLT3-ITD F691L mutant. BaF3 cells harboring FLT3-ITD and FLT3-ITD F691L were seeded and treated with crenolanib (0.2-100 nM) or with MPC-0767 (20-10000 nM) for 72 hours before cell viability was assessed using CellTiter-Glo®. EC₅₀ values were calculated using equation 1 (as described above). FIG. 11B shows that cells harboring the FLT3-ITD-F691L mutant conferred approximately 23-fold resistance to crenolanib as compared to the cells harboring FLT3-ITD (FLT3-ITD EC₅₀=4 nM versus FLT3-ITD-F691L EC₅₀=90 nM). In contrast, FIG. 11C shows that MPC-0767 had similar anti-leukemic activity against the two FLT3-ITD mutant cell lines (FLT3-ITD EC₅₀=497 nM versus FLT3-ITD-F691L EC₅₀=391 nM).

Taken together, these data demonstrate that MPC-0767 is effective at targeting kinase-resistant mutants of FLT3.

### Example 9: MPC-0767 Blocks IFN-γ-Induced PD-L1 Expression in Primary AML Samples

Interferon gamma (IFN-γ) has been shown to induce the protein expression of programmed death-ligand 1 (PD-L1) in a variety of cancer cell types, thus providing another mechanism by which tumor cells can evade the immune system.

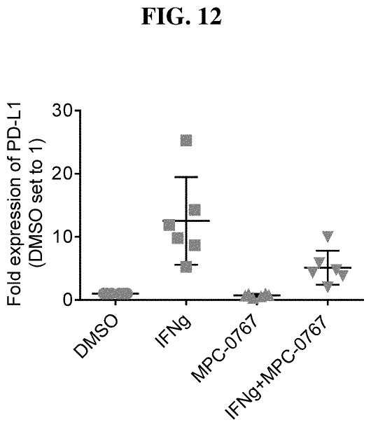

To study whether MPC-0767 blocks IFN-γ-induced PD-L1 expression, six AML patient samples harboring FLT3 WT (n=2) or FLT3-ITD (n=4) were treated with human IFN-γ (50 ng/ml) alone, MPC-0767 (1 μM) alone or the combination of the two for 24 hours. Cells were then harvested to assess PD-L1 cell surface expression by flow cytometry. Cells were also stained with the AML blast markers CD34 or CD45 (to gate on the blast population) and a viability stain to gate on viable cells. As shown in FIG. 12, all patient samples responded to IFN-γ treatment by increasing the amount of PD-L1 on their cell surface (5-25 fold). While MPC-0767 alone did not significantly reduce basal PD-L1 cell surface expression, in combination with IFN-γ, MPC-0767 significantly reduced the IFN-γ-induced PD-L1 cell surface expression (P=0.04).

This data shows that in addition to MPC-0767 possessing cytotoxic activity against FLT3-ITD AML (see above), MPC-0767 also possesses immuno-modulatory activity through abrogation of IFN-γ-induced PD-L1 expression in primary AML samples.

### Example 10: MPC-0767 Exhibits Synergistic Cytotoxic Activity

To determine whether MPC-0767 exhibits synergistic anti-proliferative activity with additional drugs, we tested it in combination with drugs that are either approved or being clinically evaluated for the treatment of AML.

Three cell lines which harbor FLT3-ITD were used for the drug combination studies (MV-4-11, MOLM-13, and MOLM-14). Cells were treated with 8 concentrations of MPC-0767 (78-10000 nM) alone, 8 concentrations of the AML drug alone (concentration ranges below), or the combination of the two (8×8). The AML combination drugs tested were: daunorubicin (0.8-100 nM); cytarabine (78-10000 nM); gilteritinib (0.8-100 nM); crenolanib (0.8-100 nM); sorafenib (0.8-100 nM); midostaurin (0.8-100 nM); or venetoclax (0.8-100 nM).

Cells were treated with the drugs (single agent or combination) for 72 hours. Drug combination activity was determined by first measuring cell viability with CellTiter-Glo®, followed by the calculation of the EC₅₀ corresponding to single agent activity, using the R DRC package (R Core Team, 2017). The combination index (CI) values were computed using the Chou-Talalay method (Chou T, Cancer Research., 2010 70(2): 440-6.), based on the viability of each drug alone and in combination, across all concentrations tested. In brief, CI was defined as:

$$CI = \frac{D 1}{D 1 alone} + \frac{D 2}{D 2 alone} \qquad ( Eq \text{-} 2 )$$

with:
D1 and D2 being the doses of Drug1 and Drug2 in the combination treatment (respectively) that give viability V.
D1alone and D2alone being the doses of Drug1 and Drug2 (respectively) as a single agent that would give the same viability V as that of the combination.
D1alone and D2alone were estimated from the Hill's equation:

$$Dalone = E C 50 * {( \frac{1 - V}{V} )}^{\frac{1}{Hill}} \qquad ( Eq \text{-} 3 )$$

with EC₅₀ and Hill being the EC₅₀ and Hill slope corresponding to Drug1 or Drug2 fitted viability curve.

Drug combinations with CI values>1 are considered antagonistic, CI values=1 are considered additive, while CI values<0.9 are considered synergistic. As additional criteria, only CI values with viability of 0.25 or lower were taken into consideration. The best combination treatment exhibiting synergy was then selected based on the maximum difference of expected versus observed viability and the lowest CI values.

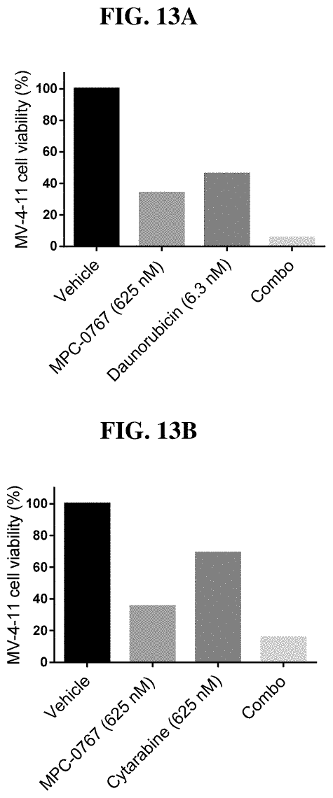

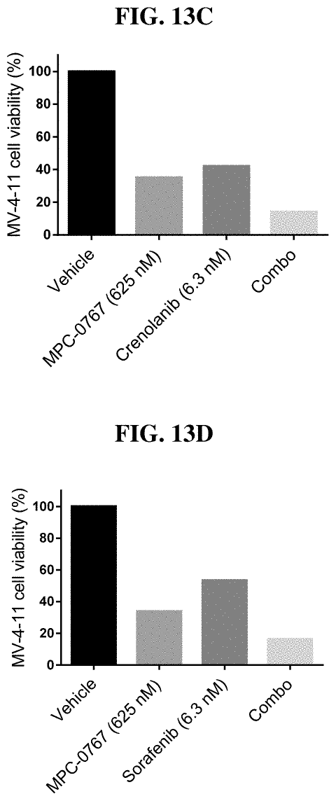

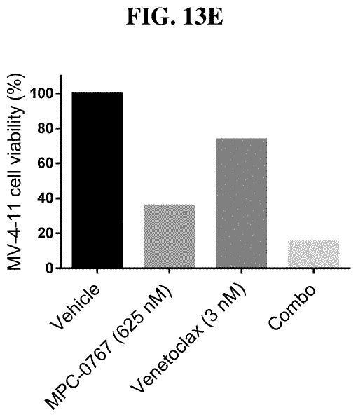

FIG. 13 shows representative synergy data in the MV-4-11 cell line treated with MPC-0767 in combination with daunorubicin (FIG. 13A), cytarabine (FIG. 13B), crenolanib (FIG. 13C), sorafenib (FIG. 13D), and venetoclax (FIG. 13E). Each graph shows the viability of cells treated with vehicle (DMSO, set to 100%), MPC-0767 alone, AML drug alone and the combination of MPC-0767+AML drug.

Table 1 shows synergistic activity of MPC-0767 (average CI values) in MV-4-11, MOLM-13 and MOLM-14 cell lines (n=2 independent experiments for each cell line except where indicated by asterisk n=1). In MV-4-11 cells, MPC-0767 is highly synergistic with daunorubicin (CI=0.6) and venetoclax (CI=0.7) and synergistic with cytarabine, crenolanib and sorafenib. In MOLM-13 cells, MPC-0767 is highly synergistic with venetoclax (CI=0.3) and less synergistic with daunorubicin, crenolanib, and gilteritinib. MPC-0767 is synergistic with venetoclax, daunorubicin, and cytarabine in MOLM-14 cells.

TABLE 1
Synergistic activity of MPC-0767 in combination
with AML drugs in AML FLT3-ITD cell lines.

| | MV-4-11 | MOLM-13 | MOLM-14 |
|---|---|---|---|
| Daunorubicin | CI = 0.6 | CI = 0.9 | CI = 0.8* |
| Cytarabine | CI = 0.8 | | CI = 0.9* |
| Crenolanib | CI = 0.7 | CI = 0.9 | |
| Sorafenib | CI = 0.8 | | |
| Gilterinib | | CI = 0.9 | |
| Venetoclax | CI = 0.7 | CI = 0.3 | CI = 0.6 |

Taken together, these data demonstrate that the HSP90 inhibitor MPC-0767 exhibits cytotoxic activity in AML cells harboring FLT3 ITD mutations. Moreover, MPC-0767 shows synergistic activity with FLT3 inhibitors in AML cells harboring FLT3 ITD mutations. Hence, HSP90 inhibitors such as MPC-0767 alone, or in combination, may have clinical efficacy in patients with AML that harbor activating mutations in FLT3.

### Example 11: MPC-0767 Exhibits Potent Anti-Tumor Activity in Combination with Venetoclax

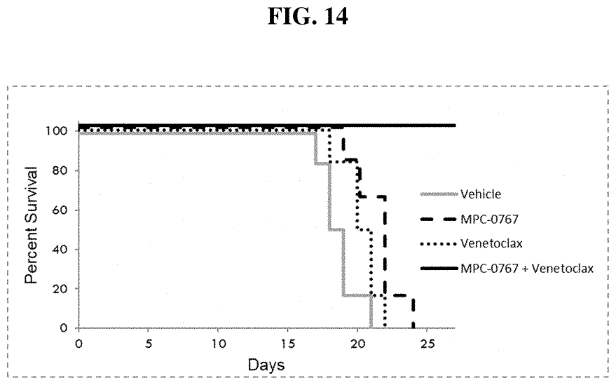

To test MPC-0767 combinatorial activity with venetoclax in vivo, a systemic survival xenograft study was performed using the MOLM-13 FLT3-ITD harboring AML cell line. Before tumor cell inoculation, NOD/SCID mice were pre-treated for 2 days with a daily intraperitoneal injection of 100 mg/kg cyclophosphamide to facilitate engraftment of the human MOLM-13 tumor cells. After the injection of cyclophosphamide, the animals were allowed to recover for 24 hours prior to inoculation with human MOLM-13 tumor cells. Each mouse was then inoculated with 1×10⁷ MOLM-13 cells in 100 μL PBS via intravenous tail vein injection. Mice were next randomized into 4 groups of 6. Three days after tumor inoculation, the mice were dosed with vehicle, MPC-0767 100-60 mg/kg QD×24 (100 mg/kg QD×6, 87.5 mg/kg QD×4, 75 mg/kg QD×3, 67.5 mg/kg QD×1, 60 mg/kg QD×10), venetoclax 45-33.8 mg/kg QD×24 (45 mg/kg QD×6, 39.4 mg/kg QD×4, 33.8 mg/kg QD×14) or the combination of MPC-0767 and venetoclax and monitored for survival. Viability and body weight loss were monitored daily. Average body weight loss did not exceed 11% in the combo group during the course of the study. As shown in FIG. 14, MPC-0767 as a single agent significantly increased median survival by 3.5 days (P<0.01, Log Rank, (Mantel Cox) test). Importantly, the combination of MPC-0767 and venetoclax resulted in 100% survival, thus providing a significantly increased median survival compared to the vehicle and both single agent arms (P<0.001, Log Rank, (Mantel Cox) test). Together this data demonstrates that MPC-0767 potently combines with venetoclax in vivo.

### Example 12: Acquired Resistance to Venetoclax in FLT3-ITD AML Cells Does Not Diminish Sensitivity to MPC-0767

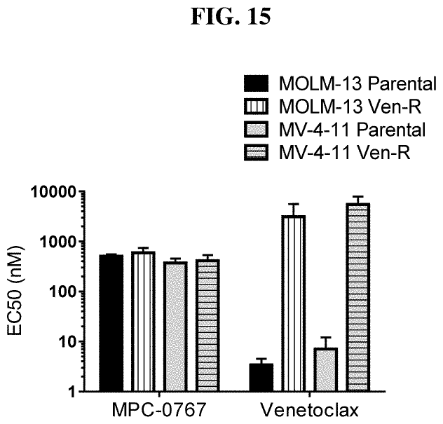

Resistance to the Bcl-2 specific inhibitor venetoclax can occur due to increased MCL-1 protein expression (Pan et al., 2017 Cancer Cell 32(6): p. 748-760 e6), thus limiting its clinical efficacy. To test the effects of acquired resistance to venetoclax on MPC-0767 sensitivity, we tested venetoclax-resistant cell lines generated from two parental FLT3-ITD AML cell lines as described by Pan et al., 2017. The parental cell lines were MOLM-13 and MV-4-11 cells. The venetoclax-resistant cell lines are designated MOLM-13 Ven-R and MV-4-11 Ven-R, respectively, in FIG. 15. As shown in the figure, MOLM-13 Ven-R and MV-4-11 Ven-R cells were highly resistant to venetoclax compared to the parental cells, as evidenced by their increased EC₅₀ values in a viability assay following 72 hours of treatment. In contrast, both parental and venetoclax-resistant cells had similar sensitivity to MPC-0767. These results indicated that the factors conferring resistance to venetoclax did not diminish the cells' sensitivity to the cytotoxic activity of MPC-0767.

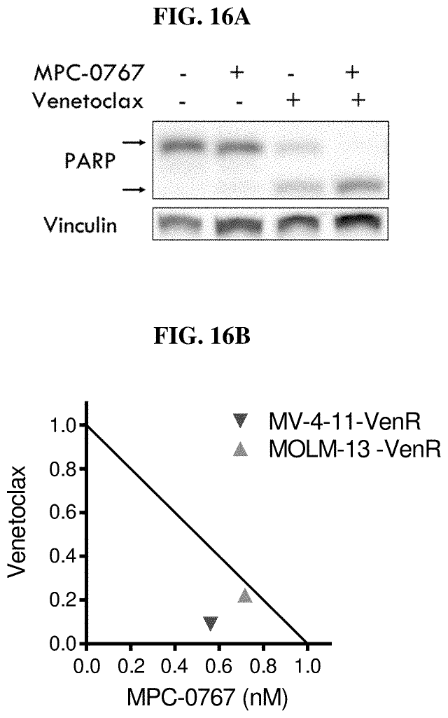

We next looked at a molecular marker of apoptosis, PARP cleavage, in the MV-4-11 Ven-R cells. Cells were treated either with MPC-0767, venetoclax, or a combination of MPC-0767 and venetoclax for 24 hours and then lysates were examined by Western analysis for full length PARP and cleaved PARP, a marker of apoptosis. As shown in FIG. 16A, Western blot analysis detected complete PARP cleavage only in cells treated with the combination of MPC-0767 and venetoclax. These data indicated that the combination was effective to induce apoptosis in these venetoclax-resistant cells.

The synergistic effects of the combination were confirmed using isobologram analysis (Tallarida, 2006 J Pharmacol Exp Ther, 319(1):1-7). The resistant cell lines, MOLM-13 Ven-R and MV-4-11 Ven-R, were treated with MPC-0767, venetoclax, or a combination of MPC-0767 and venetoclax for 72 hours and viability was assessed using the CellTiter-Glo® assay. Normalized isobolograms were used to depict drug interaction across the different cell lines and conditions, at a dose effect of 75% (EC75). In brief, the absolute EC75 for each single agent and drug combination was calculated using the R package DRC. (Ritz, C., et al. 2015 PLoS One 10(12):e0146021; and Team R. C. 2017 A language and environment for statistical computing. R Foundation for Statistical Computing, Vienna, Austria, 2017). The EC75 of the drug combination was normalized with respect to the corresponding single agent EC75 values. In cases when single agent treatments did not reach EC75, then the relative EC75 was used based on the projected value of the fitted drug response curve. When the relative EC75 was higher than the maximum concentration tested, we used the maximum concentration tested as the default value, to allow analysis across all drugs and conditions. As shown in FIG. 16B, data points for both venetoclax-resistant cell lines were below the line of additivity (diagonal line), indicating combination index values<1 and confirming synergy of the combination treatment.

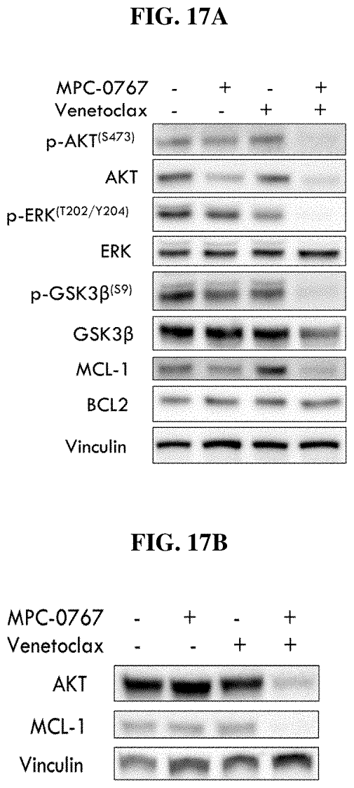

To explore the mechanism underlying MPC-0767 and venetoclax synergy, we focused on MCL-1 since its increased abundance confers resistance to venetoclax. AKT regulates the activity of GSK3 β through phosphorylation on a residue denoted serine 9 (S9). When this site is phosphorylated by AKT, GSK3 β activity is inhibited. However, inhibition of AKT prevents S9 phosphorylation, leading to GSB3β activation and subsequent degradation of MCL-1 (Lu et al., 2015 Med Oncol, 2015. 32(7): p. 206). These proteins and their phosphorylation status were examined in the MOLM-14 and MV-4-11 cell lines treated with MPC-0767, venetoclax, or a combination of MPC-0767 and venetoclax. FIG. 17A shows MOLM-14 cells treated with MPC-0767 (1 μM), venetoclax (20 nM) or the combination for 24 hours. Only the combination treatment resulted in loss of pAKT^{(S473)}, degradation of AKT and subsequent loss of GSK3β^{(Ser9)} phosphorylation. These findings are consistent with our proposal that targeting BCL-2 (e.g., with venetoclax) and at the same time targeting MCL-1 (with MPC-0767) results in synergistic cell death in FLT3-ITD AML cells. In addition, in the MV-4-11 venetoclax-resistant cell lines treated with MPC-0767 alone, venetoclax alone, or the combination, there was also reduced expression in AKT and MCL-1 by the combination of MPC-0767 and venetoclax (FIG. 17B), confirming a consistent mechanism of action for the synergy observed with MPC-0767 and venetoclax.

### Example 13: Biomarkers for MPC-0767 Efficacy in AML

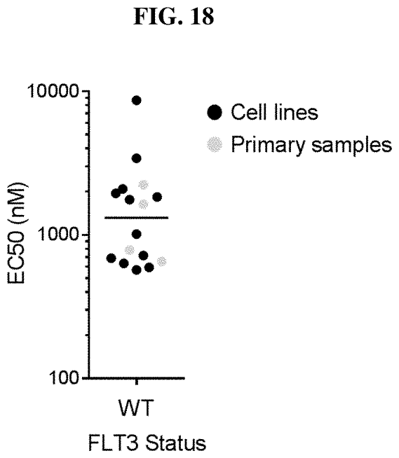

To determine whether MPC-0767 is efficacious against non-FLT3-ITD AML cells, we tested a panel of FLT3-wild type (FLT3-WT) AML cell lines and primary AML blasts for sensitivity to MPC-0767. Cells were treated with MPC-0767 for 72 hours before determining cell viability using the CellTiter-Glo® assay. EC₅₀ values were determined for all samples and shown in FIG. 18. We defined a cut-off for sensitivity at 1 μM, where cell lines having EC₅₀ values below 1 μM were considered to be sensitive, and EC₅₀ values above 1 μM considered resistant. Indeed, 6/12 cell lines and 2/4 primary cell lines displayed sensitivity (EC₅₀ value less than 1 μM).

To explore whether any mutations correlated with MPC-0767 sensitivity, we performed a statistical analysis based on the Fisher's exact test over all mutated genes in FLT3-WT AML cell lines. The processed exome sequencing data was extracted from the COSMIC Cell Line Project database and genes mutated in at least one cell line were included. The Fisher's exact test was applied to the frequency of mutated and wild type alleles observed in sensitive and resistant FLT3-WT AML cell lines. The frequency was calculated based on the number of sensitive or resistant lines containing either the mutated or wild type allele for specific genes. This rendered a 4×4 contingency table that was used to test the hypothesis of whether a mutated gene was associated with MPC-0767 sensitivity in the FLT3-WT AML cell lines.

Results from this analysis showed that RAS mutations were associated with resistant FLT3-WT AML cell lines, in a statistically significant manner (Fisher's test p-value=0.0019). FLT3-WT AML cell lines carried activating mutations in both NRAS and KRAS (Table 2), with specific mutations previously reported to stimulate MAPK signaling.

TABLE 2
Summary of AML cell lines tested for MPC-0767 sensitivity
after treatment for 72 h and cell viability determined
using CellTiter-Glo ®. Details of NRAS or KRAS
mutations in the tested cell lines are shown

| | EC50 | MPC-0767 | NRAS | KRAS |
|---|---|---|---|---|
| Cell Line | (nM) | Sensitivity | mutation? | mutation? |
| MOLM16 | 367 | Sensitive | — | — |
| TUR | 550 | Sensitive | — | — |
| OCIAML2 | 633 | Sensitive | — | — |
| ML2 | 1031 | Resistant | — | p.A146T |
| NOMO1 | 1449 | Resistant | — | p.G13D |
| OCIAML3 | 1809 | Resistant | p.Q61L | — |
| HL60 | 1957 | Resistant | p.Q61L | — |
| ME1 | 3425 | Resistant | p.Q61H | — |
| THP1 | 10000 | Resistant | p.G12D | — |

These findings suggest that mutations in key proteins, such as RAS, impact the sensitivity to MPC-0767 and further indicate that the combination of MPC-0767 and inhibitors of RAS signaling, e.g., Raf inhibitors, MEK inhibitors and ERK inhibitors, may overcome additional resistance pathways in AML cells. These findings suggest rational drug combinations that may overcome resistance pathways and restore sensitivity to MPC-0767.

### Example 14: Genome-Wide CRISPR Screen Identifies Epigenetic Regulation as a Determinant of MPC-0767 Sensitivity

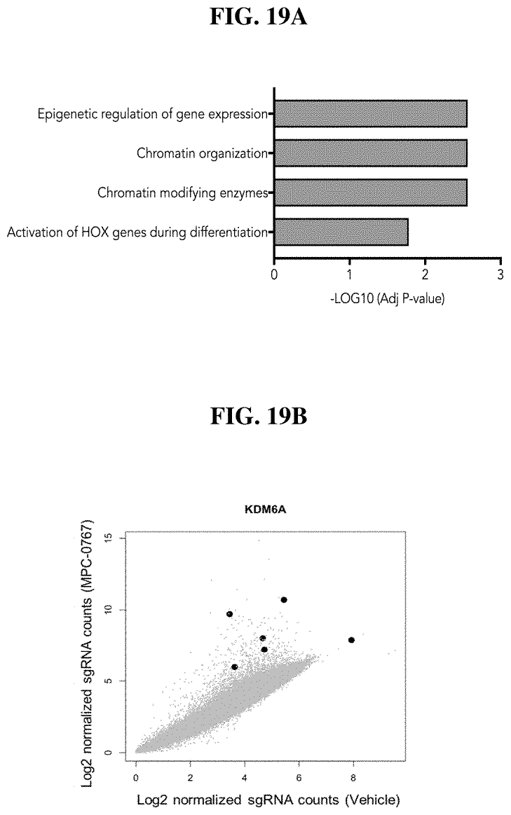

To identify genes that confer resistance to MPC-0767 upon deletion, we conducted a CRISPR-mediated genome-wide loss-of-function screen in the MOLM-14 cell line grown in the presence of 1 μM MPC-0767. We used the GeCKO V2 library (Shalem, O., et al. 2014 Science 343(6166):84-87) to perform this genetic screen. Genomic DNA harvested from surviving cells was analyzed for the identification of enriched single-guide RNAs (sgRNAs) across both GeCKO sublibraries. Gene ontology analysis of the top 20 enriched hits across both GeCKO sublibraries identified epigenetic regulation, chromatin organization and chromatin modifying enzymes as the most highly enriched pathways in the pools surviving MPC-0767 treatment (FIG. 19A).

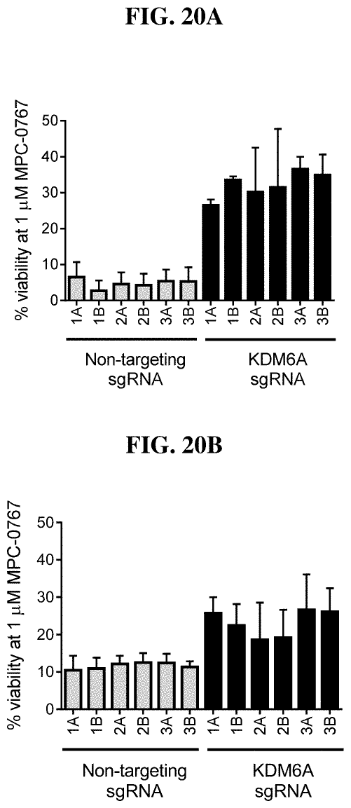

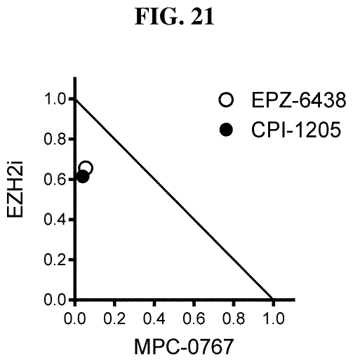

The most enriched gene from the screen was KDM6A, a histone H3K27 demethylase (Lee et al., 2007 Science 318 (5849): 447-50) (FIG. 19B). Loss of function mutations of KDM6A are observed in FLT3-ITD AML (Garg et al., 2015 Blood 126 (22):2491-501). CRISPR-mediated targeting of KDM6A with three independent sgRNAs conferred resistance to MPC-0767 in the MOLM-14 and MV-4-11 cell lines (FIG. 20A-B). To therapeutically exploit this finding, we hypothesized that inhibiting EZH2, the histone H3K27 methyltransferase that functionally opposes KDM6A, would enhance sensitivity to MPC-0767. To test this hypothesis, a FLT3-ITD cell line (MV-4-11) was used and treated with either of the two clinical stage EZH2 inhibitors, EPZ-6438 and CPI-1205, at 8 different concentrations for 4 days. Following this time, cells were counted, reseeded and treated with 8 concentrations of EZH2 inhibitor alone, 8 concentrations of MPC-0767 alone, or the combination of the two (64 total combinations). After combination treatment for 3 days, cell viability was determined using CellTiter-Glo®. Isobologram analysis was performed and data points for the combination of EPZ-6438 and MPC-0767 and of CPI-1205 and MPC-0767 were below the line of additivity (diagonal line), indicating combination index values <1 and confirming synergy of the combination treatment (FIG. 21). These findings demonstrate that epigenetic regulators can influence MPC-0767 sensitivity, that loss of function mutations in such genes may be useful as biomarkers of MPC-0767 activity, and that clinical stage compounds targeting epigenetic regulators may be combined with MPC-0767 for therapeutic use.

### Example 15: MPC-0767 Synergy with Arsenic Trioxide in AML Cell Lines

Acute promyelocytic leukemia (APL) is a subtype of acute myeloid leukemia harboring a characteristic chromosomal translocation t(15;17) which generates a fusion of the promyelocytic leukemia (PML) and retinoic acid receptor-alpha (RARα) (PML-RARα). The resulting fusion protein has an altered transcriptional profile leading to a block in cell differentiation. Agents that degrade the aberrant fusion protein including all-trans retinoic acid and arsenic trioxide (ATO) have proven effective for APL (reviewed in McCulloch et al., 2017). Intriguingly, ATO exhibits anti-proliferative activity in cells not harboring PML-RARα, suggesting it may exert additional activities that lead to cancer cell death (Miller et al., 2002). ATO has thus been evaluated in a number of heme indications that do not harbor PML-RARα (Bonati et al., 2006). Recent studies have demonstrated that the combination of ATO and sorafenib is synergistic in FLT3-ITD AML cell lines (Wang et al., 2018). One mechanistic explanation for the synergy observed was that ATO reduced the interaction between FLT3-ITD and HSP90. As a result, FLT3-ITD undergoes degradation which eliminates FLT3-ITD oncogenic signaling and tumor cells die (Wang et al., 2018). Thus, the combination with sorafenib (FLT3 inhibitor) should result in a more complete abrogation of FLT3 signaling via direct inhibition (sorafenib) and degradation (ATO). In addition, using a semi-mechanistic pharmacodynamic model which explored the concentration relationship between ATO and 1^{st} generation HSP90 inhibitors, Wetzler and colleagues (Wetzler et al., 2007) demonstrated synergy in AML cell lines with constitutive STAT3.

To test whether the combination of MPC-0767 was synergistic with ATO, we tested a panel of AML cell lines. The cell lines include those harboring FLT3-ITD (MOLM-13, MOLM-14 and MV-4-11) or FLT3 WT (ME-1, THP-1, OCI-AML-2, HL60, NOMO-1, TUR and ML-2). The cell lines were treated with 8 concentrations of MPC-0767 (234-4000 nM; 1.5 fold dilutions) alone, 8 concentrations of ATO (78-10000 nM; 2 fold dilutions) alone, or the combination of the 2 (64 data points).

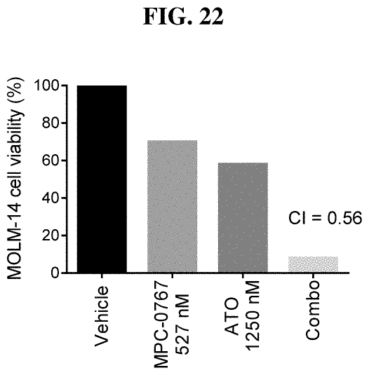

After combination treatment for 3 days, cell viability was determined using CellTiter-Glo®. Combination index (CI) values were calculated for each cell line using the Chou-Talalay equation, where CI values <1 denotes synergy, CI=1 denotes additivity and CI>1 denotes antagonism. An example is shown in FIG. 22 where MOLM-14 cells were treated with MPC-0767 (527 nM), ATO (1250 nM) or the combination (combo). Importantly, the combination reduced viability greater than the additive effect of either agent alone and a retrieved a CI value of 0.56, confirming synergy. Table 3 shows the CI values for all the cell lines tested and the specific concentration of MPC-0767 and ATO. Synergy was observed in all cell lines tested. These findings establish that the combination of MPC-0767 is synergistic with ATO in AML cells. Moreover, synergy was observed at clinically relevant concentrations of MPC-0767 both in cell lines harboring the FLT3-ITD mutation and those that did not.

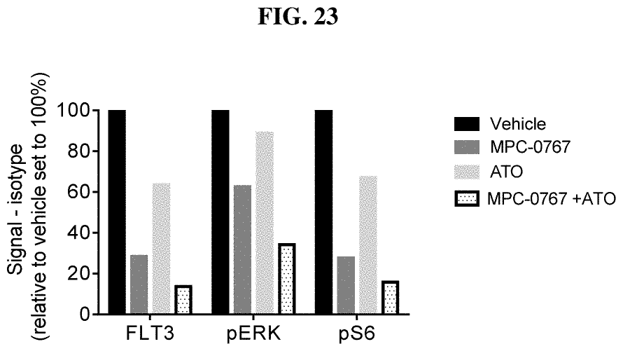

We next explored whether the synergistic activity of MPC-0767 and ATO was due to a more complete abrogation of FLT3-ITD oncogenic signaling. MOLM-13 cells were treated with MPC-0767 (800 nM), ATO (625 nM) or the combination for 24 hours. After this time cells were harvested for the assessment of cell surface FLT3 expression by flow cytometry. To additionally measure the effects of abrogating FLT3, we assessed phospho-ERK (pERK) and phospho-S6 (pS6), as these are two known downstream effectors. As shown in FIG. 23, MPC-0767 and ATO as single agents reduced FLT3, pS6 and mildly reduced pERK. However, the combination resulted in a greater reduction of each protein or phosphoprotein compared to either agent alone. These findings suggest that the synergistic anti-proliferative effect observed in FLT3-ITD AML cell lines is manifested at least in part through a more complete inhibition of FLT3 oncogenic signaling.

TABLE 3
Summary of combination index (CI) values obtained
for the combination of MPC-0767 and ATO in all AML
cell lines tested. CI values < 1 denote synergy.

| | | Combination | | |
|---|---|---|---|---|
| | | MPC-0767 | ATO | CI |
| Cell line | FLT3-ITD? | conc. (nM) | conc. (nM) | value |
| MOLM-13 | Yes | 790 | 625 | 0.65 |
| MOLM-14 | Yes | 527 | 1250 | 0.56 |
| MV-4-11 | Yes | 790 | 625 | 0.71 |
| OCI-AML-2 | No | 790 | 1250 | 0.67 |
| NOMO-1 | No | 1185 | 2500 | 0.69 |
| ML2 | No | 790 | 1250 | 0.54 |
| TUR | No | 527 | 5000 | 0.66 |
| HL-60 | No | 790 | 5000 | 0.85 |
| ME-1 | No | 1185 | 10000 | 0.21 |
| THP-1 | No | 2667 | 10000 | 0.12 |

### Example 16: MPC-0767 Overcomes Alternate Pathway Activation that Confers Resistance to FLT3 Inhibitors

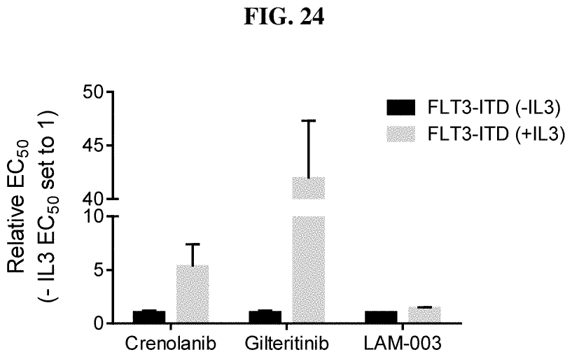

Conditions that mimic stromal signaling in the bone marrow can confer resistance to FLT3 inhibitors through the activation of alternative cell surface receptors (Karjalainen et al., 2017). The BaF3 cell system was used to test MPC-0767 efficacy under conditions that confer resistance to FLT3 inhibitors. BaF3 cells require the supplementation of IL-3 to activate the IL-3 receptor for growth. However, in BaF3 cells transfected with FLT3-ITD, cells no longer require IL3 as survival is solely driven by oncogenic FLT3 signaling. As such, FLT3-ITD expressing cells in the absence of IL-3 are sensitive to FLT3 inhibition by the FLT3 inhibitors gilteritinib or crenolanib (FIG. 24). However, the addition of IL3 activates an alternative, non-FLT3-dependent pro-survival pathway, such that cells are rendered resistant to FLT3 inhibitors (Sung et al., 2017). In contrast, BaF3 expressing FLT3-ITD and treated with or without exogenous IL3 are equally sensitive to MPC-0767 (FIG. 24). These findings demonstrate that MPC-0767 can inhibit multiple pro-survival pathways.

### Example 17: MPC-0767 Exhibits Enhanced Anti-Tumor Activity in Combination with 5′ azacitadine

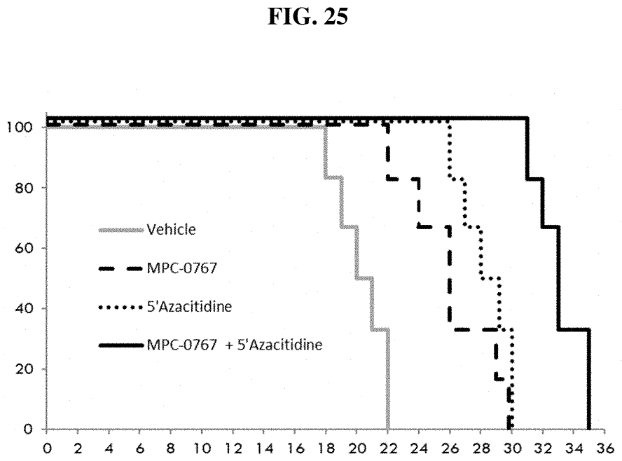

To test MPC-0767 in combination with 5′azacitadine in vivo, a systemic survival xenograft study was performed using the MOLM-13 FLT3-ITD harboring AML cell line. Before tumor cell inoculation, NOD/SCID mice were pre-treated for 2 days with a daily intraperitoneal injection of 100 mg/kg cyclophosphamide to facilitate engraftment of the human MOLM-13 tumor cells. After the injection of cyclophosphamide, the animals were allowed to recover for 24 hours prior to inoculation with human MOLM-13 tumor cells. Each mouse was then inoculated with 1×10⁷ MOLM-13 cells in 100 μL PBS via intravenous tail vein injection. Mice were next randomized into 4 groups of 6 mice each. Three days after tumor inoculation, the mice were dosed with vehicle, MPC-0767 75 mg/kg (QD×5; 1 day off; Q×26 p.o.); 5′azacitidine 2 mg/kg (QD×4 i.p.) or the combination of MPC-0767 and 5′azacitidine (treated as for single agents) and monitored for survival. Viability and body weight loss were monitored daily. Average body weight loss did not exceed 11% in the combination group during the course of the study. As shown in FIG. 25, MPC-0767 and 5′azacitidine as single agents significantly increased median survival of the mice by 5.5 days and 8 days respectively (P<0.01 and P<0.001 respectively, Log Rank, (Mantel Cox) test). Importantly, the combination of MPC-0767 and 5′azacitidine resulted in significantly increased median survival compared to the vehicle and both single agent arms (P<0.001, Log Rank, (Mantel Cox) test). These findings demonstrate that the combination of MPC-0767 and 5′azacitidine has anti-leukemic activity and may be an effective therapy for FLT3-ITD AML patients.

### Example 18: MPC-0767 Enhances T Cell-Mediated Lilling of AML Cells

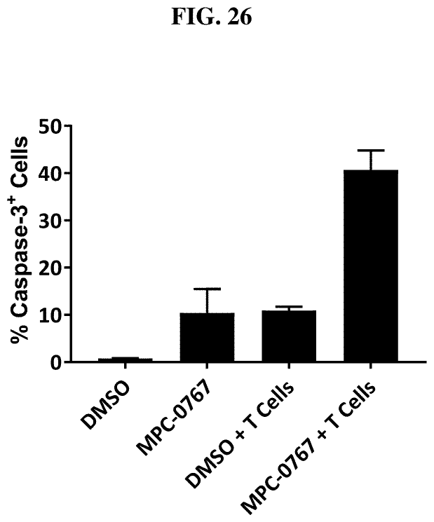

The ability of MPC-0767 to increase T cell killing was determined in an in vitro T-cell-mediated killing assay. The OCI-AML2 AML cell line was labeled with the cell staining dye CFSE and treated overnight with MPC-0767 (2 μM) and human cytomegalovirus pp65₄₉₅₋₅₀₃ peptide. OCI-AML2 cells were washed to remove MPC-0767 and peptide and then co-cultured with a T cell line enriched for pp65-specific CD8⁺ T cells at an approximate ratio of 2.5:1 (T cells:OCI-AML2). After 4 hours of co-culture, cells were harvested, fixed, permeabilized, and stained for the active form of caspase-3 as a direct read-out of apoptotic cell death. The percent active caspase-3+ out of all CFSE+ cells (OCI-AML-2 cells only) are shown in FIG. 26. A synergistic increase (Combination Index (CI), of 0.53) in apoptotic cells was observed with the combination of MPC-0767 and pp65 enriched CD8⁺ T cells. These findings demonstrate that MPC-0767 can alter tumor cells rendering them more vulnerable to T cell-mediated killing.

The CI is a quantitative measure used to determine whether the combined effect of a drug pair is synergistic, additive, or antagonistic. The CI is calculated as CI=(E1+E2)/E12, where E12 is a normalized biological response (e.g., % Caspase-3+ cells) for the combination of Drug A and Drug B, and E1 and E2 are the response measured for each single drug treatment, respectively. CI values less than 1 indicate synergy, with the magnitude of the effect indicated by how much less than 1 the synergy score is. A more detailed mathematical treatment of this relationship is described in Shin et al. 2018.

### Example 19: MPC-0767 Demonstrates in Vivo Efficacy in the Immunocompetent MC38 Syngeneic Model

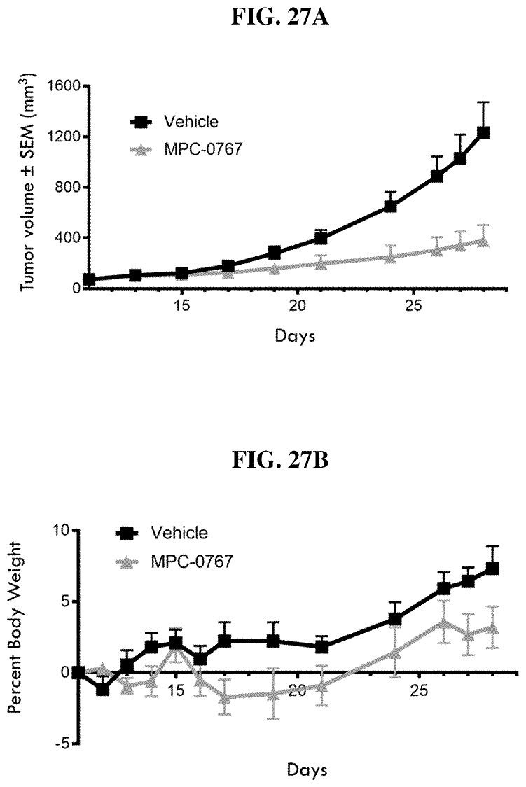

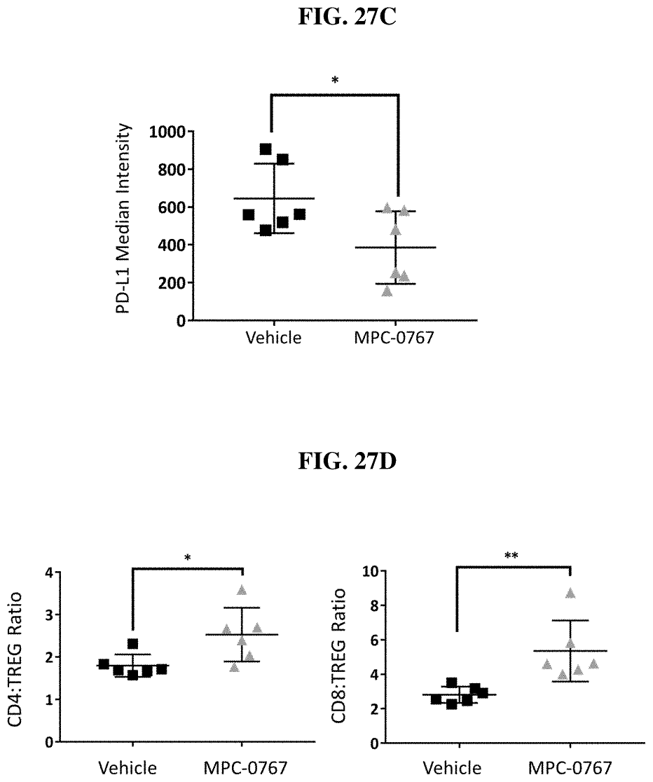

To demonstrate MPC-0767 efficacy in an in vivo model with an intact immune system, a syngeneic study was performed using the murine MC38 colon cancer cell line. Each C57BL/6 mouse was inoculated subcutaneously in the right flank with 2.5×10⁵ tumor cells in 0.1 ml PBS. When the mean tumor volume reached 73 mm³ in size, mice were randomized into 2 groups of 6. Mice were then dosed orally with either vehicle or 150 mg/kg MPC-0767 QD×17. Tumor measurements (caliper) were taken on the indicated days. As shown in FIG. 27, MPC-0767 induced a tumor growth inhibition of 69.5% (FIG. 27A), without significant effects on body weight (FIG. 27B). Student t-test was used to evaluate the statistical significance of the difference between these groups, P=0.01. This data confirms that MPC-0767 displays anti-tumor activity in an in vivo syngeneic model.

To test if MPC-0767 may induce an anti-tumor immune response in addition to direct cytotoxic activity, down regulation of PD-Ll and the effector/regulatory T-cell ratio was measured in the same MC38 syngeneic model. On day 21, when the average tumor volume was 372 mm³ in size, a second group of mice (n=6) was treated with 150 mg/kg MPC-0767 QD×7. One day post the last dose (day 28 post inoculation) tumors were harvested from the vehicle and 150 mg/kg MPC-0767 QD×7 group. Tumor infiltrating leukocytes (CD45⁺, CD3⁻) within the dissociated tumors were analyzed for PD-L1 expression by flow cytometry. A significant reduction of PD-L1 was observed indicating that MPC-0767 can repress this immunosuppressive ligand in vivo (FIG. 27C). To assay the effects of this repression on immune cell populations within MC38 tumors the ratio of CD4⁺ (CD45⁺, CD3⁺, CD4⁺) and CD8⁺ T-cells (CD45⁺, CD3⁺, CD4⁻) to regulatory T-cells (CD45⁺, CD3⁺, CD4⁺, FOXP3⁺) was also assessed by flow cytometry. A significant increase of the CD4:TREG and CD8:TREG ratio was observed in the MPC-0767 treated group (FIG. 27D), which is suggestive of an anti-tumor immune response. Together this data supports that MPC-0767 anti-tumor activity involves induction of anti-tumor immune response.

### Example 20: MPC-0767 Synergy with a MAPK Pathway Inhibitor in AML Cell Lines

The mitogen-activated protein kinase (MAPK) pathway is a critical integration point linking external stimuli at the cell survival and transducing them to intracellular signals that mediate differentiation, survival and proliferation. Indeed, AML cells targeted by selective MAPK inhibitors result in reduced cell survival (Milella et al., 2001). The combination of MPC-0767 and trametinib, a clinical stage MEK inhibitor that has been approved for the treatment of melanoma patients whose tumor harbors BRAF V600E, was tested in a panel of AML cell lines. The cell lines include those harboring FLT3-ITD (MOLM-13, MOLM-14 and MV-4-11) or FLT3 WT+RAS WT (OCI-AML-2) or FLT3 WT+RAS mutant (ML-2) The cell lines were treated with 8 concentrations of MPC-0767 (234-4000 nM; 1.5 fold dilutions) alone, 8 concentrations of ATO (0.8-100 nM; 2 fold dilutions) alone, or the combination of the 2 (64 data points).

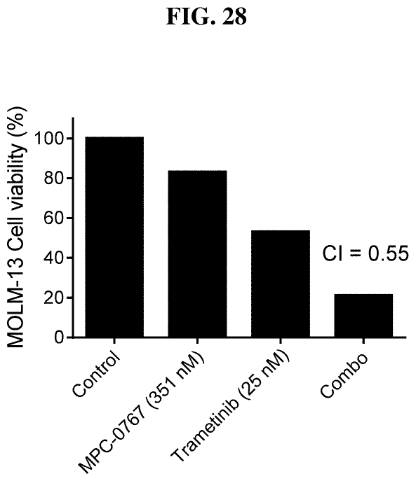

After combination treatment for 3 days, cell viability was determined using CellTiter-Glo®. Combination index (CI) values were calculated for each cell line using the Chou-Talalay equation, where CI values <1 denotes synergy, CI=1 denotes additivity and CI>1 denotes antagonism. An example is shown in FIG. 28 where MOLM-13 cells were treated with MPC-0767 (351 nM), trametinib (25 nM) or the combination (combo). Importantly, the combination reduced viability greater than the additive effect of either agent alone and a retrieved a CI value of 0.55, confirming synergy. Table 4 shows the CI values for all the cell lines tested and the specific concentration of MPC-0767 and trametinib. Moreover, synergy was observed at clinically relevant concentrations of MPC-0767 in cell lines that harbored FLT3-ITD or not, or in a cell line that harbors a RAS mutation.

TABLE 4
Summary of combination index (CI) values obtained for
the combination of MPC-0767 and trametinib in all AML
cell lines tested. CI values < 1 denote synergy.

| | | Combination | | |
|---|---|---|---|---|
| | FLT3 & RAS | MPC-0767 | Trametinib | CI |
| Cell line | status | conc. (nM) | conc. (nM) | value |
| MOLM-13 | FLT3-ITD; | 351 | 25 | 0.55 |
| | RAS WT | | | |
| MOLM-14 | FLT3-ITD; | 790 | 6.3 | 0.62 |
| | RAS WT | | | |
| MV-4-11 | FLT3-ITD; | 527 | 6.3 | 0.67 |
| | RAS WT | | | |
| OCI-AML-2 | FLT3 WT; | 790 | 0.78 | 0.64 |
| | RAS WT | | | |
| ML2 | FLT3 WT; | 790 | 100 | 0.32 |
| | RAS mutant | | | |

### Example 21: MPC-0767 Inhibition of PD-L1 Expression Increases T Cell Activation

The addition of antibodies that block the PD-1/PD-L1 pathway stimulate an increased T cell response in vitro, in pre-clinical animal models, and in cancer patients. This can lead to tumor regressions or tumor clearance in patients. To examine MPC-0767 effects on PD-L1 and T cell activation, we used a model system in which PD-1+ Jurkat T cells express luciferase under the control of the NFAT promoter (Promega, hereafter referred to Jurkat reporter cells). When T cells are stimulated through the T cell receptor (TCR), activation of the NFAT pathway drives expression of luciferase. Hence, in this model system luciferase is a surrogate marker of T cell activation.

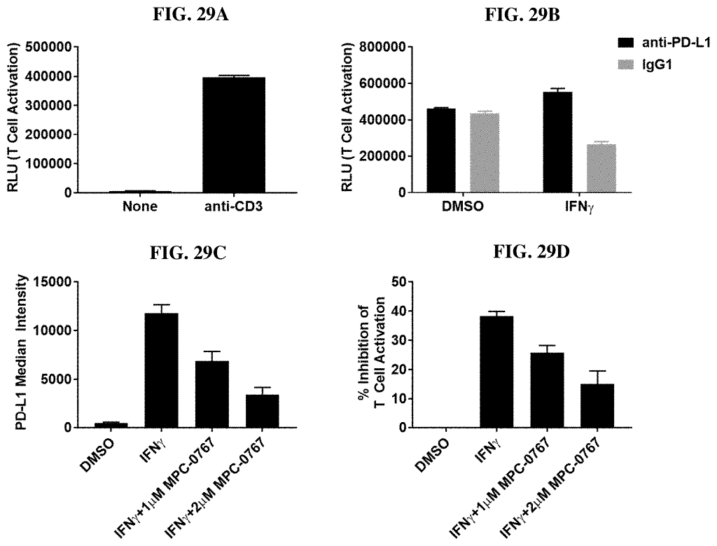

As shown in FIG. 29A, a 6 hour incubation of THP-1 AML cells with Jurkat reporter cells and low-dose anti-CD3 (10 ng/ml) leads to luciferase expression due to TCR driven activation of Jurkat reporter cells. FIG. 29B shows that THP-1 cells treated for 24 hr with IFNγ (50 ng/ml) have reduced ability to activate T cells (reduced luciferase). This can be attributed to IFNγ-mediated upregulation of PD-L1, as addition of a PD-L1 blocking antibody (atezolizulmab, 5 μm/ml) restores T cell activation to untreated levels.

We next determined whether MPC-0767 reduction of PD-L1 could increase T cell stimulation similar to anti-PD-L1 blocking antibodies. THP-1 cells were treated overnight with IFNγ in the presence or absence of MPC-0767 (1 μM or 2 uM). THP-1 cells were washed and a portion saved for flow cytometry analysis of PD-L1 expression. The remaining cells were incubated with Jurkat reporter cells and anti-CD3 (10 ng/ml) for 6 hours. MPC-0767 dose-dependently reduced PD-L1 expression on THP-1 cells (FIG. 29C). MPC-0767 was also able to dose-dependently reduce inhibition of T cell activation (FIG. 29D), demonstrating that modulation of PD-L1 expression by MPC-0767 has a functional consequence on T cell activity.

### Example 22: MPC-0767 Demonstrates Anti-Tumor Activity in a Systemic in Vivo AML Model

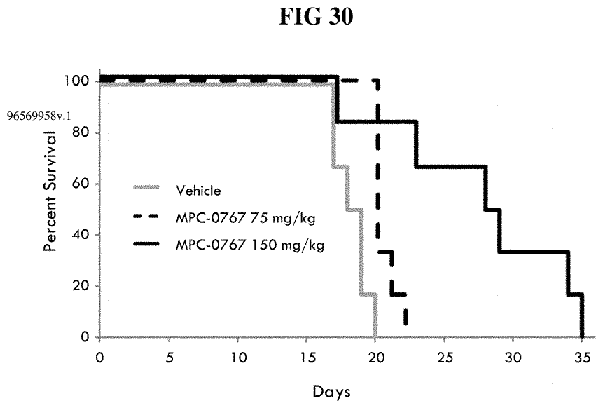

To further test MPC-0767 activity in vivo, a systemic survival xenograft study was performed using the MOLM-13 FLT3-ITD harboring AML cell line. Before tumor cell inoculation, NOD/SCID mice were pre-treated for 2 days with a daily intraperitoneal injection of 100 mg/kg cyclophosphamide to facilitate engraftment of the human MOLM-13 tumor cells. After the injection of cyclophosphamide, the animals were allowed to recover for 24 hours prior to inoculation with human MOLM-13 tumor cells. Each mouse was then inoculated with 1×10⁷ MOLM-13 cells in 100 μL PBS via intravenous tail vein injection. Mice were next randomized into 3 groups of 6. Three days after tumor inoculation, the mice were dosed with vehicle, 75 mg/kg MPC-0767 or 150 mg/kg MPC-0767 once a day and monitored for survival. Viability and body weight loss were monitored daily. Significant body weight loss and/or clinical symptoms (paralysis, hypothermia, or tachypnea) were only observed just prior to morbidity in all three groups. As shown in FIG. 30, MPC-0767 significantly increased median survival by 1.5 days at 75 mg/kg and by 10 days at 150 mg/kg (P<0.01, Log-Rank (Mantel Cox) test). In summary, MPC-0767 demonstrated significant dose-dependent anti-tumor activity.

1-12. (canceled)

13. The method of claim 31, wherein the subject is human.

14. The method of claim 31, wherein the pharmaceutical composition is adapted for oral, buccal, or parenteral administration.

15-30. (canceled)

31. A method for treating acute myelogenous leukemia (AML) in a subject in need thereof, the method comprising administering to the subject a pharmaceutical composition comprising a therapeutically effective amount of MPC-0767, or a pharmaceutically acceptable salt thereof, and optionally a pharmaceutically acceptable carrier or excipient.

32. The method of claim 31, wherein the pharmaceutical composition comprises a mesylate salt of MPC-0767.

33. The method of claim 31, wherein the AML is refractory to, or has relapsed after, treatment with at least one protein kinase inhibitor (PKI).

34. The method of claim 33, wherein the AML is refractory to, or has relapsed after, treatment with one or more of midostaurin, quizartinib, tandutinib, and sorafenib.

35. The method of claim 31, wherein the AML is refractory to, or has relapsed after, treatment with one or more of gilteritinib, crenolanib, sorafenib, midostaurin, daunorubicin, doxorubicin, epirubicin, mitoxantrone, idarubicin, and cytarabine.

36. The method of claim 31, wherein the AML is characterized as having one or more activating mutations in FLT3.

37. The method of claim 36, wherein the one or more activating mutations in FLT3 is selected from the FLT3 ITD mutation, a point mutation at FLT3 D835, a point mutation at FLT3 1836, the point mutation FLT3 N676K, and the point mutation F691L.

38. The method claim 37, wherein the one or more activating mutations in FLT3 is the FLT3 ITD mutation.

39. The method of claim 31, further comprising a step of administering one or more additional active pharmaceutical agents (APIs) to the subject.

40. The method of claim 39, wherein the one or more additional APIs is a protein kinase inhibitor (PKI), a chemotherapeutic agent, an FLT3 inhibitor, a PD-1/PD-L1 inhibitor, a Ras/Raf/MEK/ERK pathway inhibitor, a Bcl-2 pathway inhibitor, a checkpoint inhibitor, a therapeutic agent that enhances anti-tumor immunity, or an EZH2 inhibitor.

41. The method of claim 40, wherein the FLT3 inhibitor is selected from crenolanib, tandutinib, gilteritinib, midostaurin, quizartinib, and sorafenib.

42. The method of claim 40, wherein the PD-1/PD-L1 inhibitor is selected from the group consisting of AMP-224, AMP-514/MEDI-0680, atezolizumab, avelumab, BGB-A317, BMS936559, durvalumab, JTX-4014, nivolumab, pembrolizumab, and SHR-1210.

43. The method of claim 40, wherein the Bcl-2 pathway inhibitor is selected from the group consisting of ABT-737, AT-101 (Gossypol), APG-1252, A1155463, A1210477, navitoclax, obatoclax, sabutoclax, venetoclax, S 55746, WEHI-539, AMG-176, MIK665 and 5641315.

44. The method of claim 40, wherein the Bcl-2 pathway inhibitor is an inhibitor of BCL2, BCLXL, or MCL1.

45. The method of claim 40, wherein the Bcl-2 pathway inhibitor is selected from ABT-737, navitoclax, and venetoclax.

46. The method of claim 39, wherein the one or more additional APIs is selected from the group consisting of daunorubicin, doxorubicin, epirubicin, mitoxantrone, idarubicin, and cytarabine.

47. The method of claim 39, wherein the one or more additional APIs is selected from crenolanib, cytarabine, daunorubicin, gilteritinib, sorafenib, and venetoclax.

48. The method of claim 39, wherein the one or more additional APIs is venetoclax.

49-54. (canceled)

55. The method of claim 31, wherein the AML is refractory to, or has relapsed after, treatment with a Bcl-2 pathway inhibitor.

56. The method of claim 55, wherein the Bcl-2 pathway inhibitor is venetoclax.

57. The method of claim 55, further comprising administering one or more additional active pharmaceutical agents (APIs) to the subject.

58. The method of claim 57, wherein the one or more additional APIs is a protein kinase inhibitor (PKI), a chemotherapeutic agent, an FLT3 inhibitor, a PD-1/PD-L1 inhibitor, or a Bcl-2 pathway inhibitor.

59. The method of claim 58, wherein the FLT3 inhibitor is selected from crenolanib, gilteritinib, midostaurin, quizartinib, and sorafenib.

60. The method of claim 58, wherein the PD-1/PD-L1 inhibitor is selected from the group consisting of AMP-224, AMP-514/MEDI-0680, atezolizumab, avelumab, BGB-A317, BMS936559, durvalumab, JTX-4014, nivolumab, pembrolizumab, and SHR-1210.

61. The method of claim 58, wherein the Bcl-2 pathway inhibitor is selected from the group consisting of ABT-737, AT-101 (Gossypol), APG-1252, A1155463, A1210477, navitoclax, obatoclax, sabutoclax, venetoclax, S 55746, WEHI-539, AMG-176, MIK665 and 5641315.

62. The method of claim 58, wherein the Bcl-2 pathway inhibitor is an inhibitor of BCL2, BCLXL, or MCL1.

63. The method of claim 58, wherein the Bcl-2 pathway inhibitor is selected from ABT-737, navitoclax, and venetoclax.

64. The method of claim 57, wherein the one or more additional APIs is selected from the group consisting of daunorubicin, doxorubicin, epirubicin, mitoxantrone, idarubicin, and cytarabine.

65. The method of claim 57, wherein the one or more additional APIs is selected from crenolanib, cytarabine, daunorubicin, gilteritinib, sorafenib, and venetoclax.

66. The method of claim 57, wherein the one or more additional APIs is venetoclax.

67. The method of claim 31, further comprising administering a Ras/Raf/MEK/ERK pathway inhibitor.

68. The method of claim 67, wherein the RAS pathway inhibitor is selected from a Raf inhibitor such as vemurafenib, sorafenib, or dabrafenib, a MEK inhibitor such as AZD6244 (Selumetinib), PD0325901, GSK1120212 (Trametinib), U0126-EtOH, PD184352, RDEA119 (Rafametinib), PD98059, BIX 02189, MEK162 (Binimetinib), AS-703026 (Pimasertib), SL-327, BIX02188, AZD8330, TAK-733, cobimetinib or PD318088, and an ERK inhibitor such as LY3214996, BVD-523 or GDC-0994.

69. (canceled)

70. (canceled)

71. The method of claim 31, the method further comprising determining the FLT3 and RAS mutant status in a sample of AML cancer cells from the subject and treating the subject with a combination therapy comprising MPC-0767 and a Ras/Raf/MEK/ERK pathway inhibitor where the status is FLT3 normal/non-FLT3-ITD and RAS mutant.

72. The method of claim 71, wherein a status of RAS mutant is defined by the presence of one or more activating mutations in NRAS or KRAS.

73. The method of claim 71, wherein the one or more activating mutations in NRAS or KRAS is a mutation in the polynucleotide sequence encoding the RAS protein that results in an amino acid change selected from the group consisting of A146T and G13D of KRAS; or Q61L, Q61H, and G12D of NRAS.

74. The method of claim 31, further comprising administering an EZH2 inhibitor.

75. (canceled)

76. The method of claim 31, the method further comprising determining or receiving the EZH2 status of the AML in a biological sample of the AML from the subject and treating the subject with MPC-0767 therapy where the status is an EZH2 loss of function mutation, or treating the subject with a combination therapy comprising MPC-0767 and an EZH2 inhibitor where the EZH2 status is normal or a gain of function EZH2 mutation.

77. (canceled)

78. The method of claim 31, further comprising administering an EZH2 inhibitor.

79. The method of claim 78, wherein the EZH2 inhibitor is selected from GSK343, EPZ6438 (Tazemetostat), CPI-1205, GSK2816126, and PF-06821497.

80-85. (canceled)
